(19) United States
(12) Patent Application Publication
(10) Pub. No.: US 2008/0293125 A1
(43) Pub. Date: Nov. 27, 2008

(54) ENGINEERED MICROORGANISMS FOR PRODUCING ISOPROPANOL

(75) Inventors: Ezhilkani Subbian, Mountain View, CA (US); Peter Meinhold, Highlands Ranch, CO (US); Thomas Buelter, Denver, CO (US); Andrew C. Hawkins, Parker, CO (US)

Correspondence Address: PAUL, HASTINGS, JANOFSKY & WALKER LLP, 875 15th Street, NW, Washington, DC, 20005, US

(73) Assignee: Gevo, Inc., Centennal, CO (US)

(21) Appl. No.: 12/106,173

(22) Filed: Apr. 18, 2008

Related U.S. Application Data
(60) Provisional application No. 60/912,547, filed on Apr. 18, 2007.

(51) Int. Cl. C07C 31/10; C12N 1/21; C12N 1/15; C12P 7/04; C12N 1/19

(52) U.S. Cl. 435/252.3; 435/254.11; 435/254.2; 435/252.33; 435/254.21; 435/254.22; 435/254.23; 435/254.3; 435/252.31; 435/252.32; 435/252.34; 435/252.35; 568/840; 435/157

(57) ABSTRACT

In an embodiment, there is disclosed a recombinant microbial host cell having each of the DNA molecules encoding a polypeptide or group of polypeptides that catalyze the conversion:

(i) Acetyl-CoA to Acetate and CoA (conversion 1)

(ii) Acetyl-CoA to Acetoacetyl-CoA and CoA (conversion 2)

(iii) Acetoacetyl-CoA and Acetate to Acetoacetate and Acetyl-CoA (conversion 3.1)

(iv) Acetoacetate to Acetone and CO2 (conversion 4)

(v) Acetone and NAD(P)H and H+ to Isopropanol and NAD(P)+ (conversion 5)

wherein at least one DNA molecule is heterologous to the microbial host cell and wherein the microbial host cell produces isopropanol. In another embodiment, a method is disclosed for the production of isopropanol including providing a recombinant microbial host cell, the host cell of (i) with a fermentable carbon substrate in a fermentation medium under conditions whereby isopropanol is produced, and recovering the isopropanol.

This application claims the benefit of U.S. Provisional Application No. 60/912,547 filed Apr. 18, 2007, by Thomas Buelter, et al., for ENGINEERED MICROORGANISMS FOR PRODUCING ISOPROPANOL, which patent application is hereby incorporated herein by reference.

### FIELD OF THE INVENTION

The present invention relates to a process for the conversion of carbohydrates to isopropanol using microorganisms.

### BACKGROUND OF THE INVENTION

In the early 1940s, Henry Ford first investigated the use of soy based plastics in vehicles. This initiated the first wave of interest in bio-based and agri-based industrial materials. These, traditionally defined as ‘engineering material made from living matter such as starch or bio-derived monomers that is often biodegradable’, have the potential to improve sustainability of natural resources, environmental quality and national security while competing economically with petrochemically derived materials. Growing concern over depleting fossil energy resources and the geo-political instability of oil-rich nations has re-focused both government and public efforts in the area of bio-based materials, fuels and chemicals. In addition, environmental concerns relating to the possibility of carbon dioxide related climate change is an important social and ethical driving force which is starting to result in government regulations and policies such as the 2002 US Farm bill (http://www.rurdev.usda.gov/rbs/farmbill/) the goal of which is to increase the government's purchase and use of bio-based products.

Bio-based materials are starting to replace traditional petrochemically derived materials in a growing number of areas. For example, ink derived from soybean oil has replaced more than 90% of the petro-based ink used by the US newspaper industry (Wool, R P., Xiuzhi, S S. Bio-Based Polymers and Composites. (2005) Elsevier Academic Press). ‘Soy ink’ is available in brighter colors, is more environmentally friendly and allows for more efficient paper recycling. Paints, detergents and plastics based on vegetable oils and fats function as viable green alternatives to traditional petro-based ones. Poly lactic acid (PLA), made using lactate derived from corn or sugarcane, is a biodegradable polyester. The uses of bio-based PLA range from biomedical applications such as sutures and stents to packaging material and disposable tableware. Bio-propanediol, made from corn, can be used as a starting material for a number of industrial products including composites, adhesives, laminates, copolyesters and solvents. Bio-based alcohols such as isopropanol, ethanol, butanol and isobutanol offer another environmentally friendly raw material that can be used to develop greener materials, fuels and chemicals.

The first and biggest use of isopropanol (IPA) is as a solvent. The other most significant use of IPA is as a chemical intermediate. It is a component of cleaners, disinfectants, room sprays, lacquers and thinners, adhesives, pharmaceuticals, cosmetics and toiletries. It is also used as an extractant and as a dehydrating agent. Xanthan gum, for example, is extracted with IPA. In addition, isopropanol is also used as a gasoline additive, to dissolve water and ice in fuel lines and tanks thereby preventing the water from accumulating in the fuel lines and freezing at low temperatures. IPA is also sold as rubbing alcohol and used as a disinfectant.

IPA is currently produced by one of two processes that use petrochemically derived precursors: (1) a two-step (indirect) process during which propylene is hydrogenated and then hydrolysed using acid and water or (2) a one-step (direct) process during which propylene is hydrogenated using an acid catalyst. In 2003, the global petrochemical based IPA production reached 2152 thousand metric tons with most of the production focused in the US, Western Europe and Japan. The global demand for isopropanol and propylene continues to increase at a rate of about 3% per year. An environmentally friendly and bio-based alternative to the petro-based production process is the production of IPA by fermentation from renewable biomass. However, to be viable and outperform in the current petrochemical IPA market, a fermentative process for the production of IPA must be cost-effective.

### SUMMARY OF THE INVENTION

In an embodiment, an engineered microorganism is provided that produces isopropanol at high yield by biochemically converting a carbon source to isopropanol. The engineered microorganisms express a metabolic pathway for the production of isopropanol.

In an embodiment, there is provided a recombinant microbial host cell comprising each of the DNA molecules encoding a polypeptide or group of polypeptides that catalyze the conversion:

(i) Acetyl-CoA to Acetate and CoA (conversion 1)

(ii) Acetyl-CoA to Acetoacetyl-CoA and CoA (conversion 2)

(iii) Acetoacetyl-CoA and Acetate to Acetoacetate and Acetyl-CoA (conversion 3.1)

(iv) Acetoacetate to Acetone and CO2 (conversion 4)

(v) Acetone and NAD(P)H and H+ to Isopropanol and NAD(P)+ (conversion 5)

wherein at least one DNA molecule is heterologous to said microbial host cell and wherein said microbial host cell produces isopropanol.

In another embodiment, there is provided a recombinant microbial host cell comprising each of the DNA molecules encoding a polypeptide that catalyzes the conversion:

(i) Acetyl-CoA to Acetoacetyl-CoA and CoA (conversion 2)

(ii) Acetoacetyl-CoA+H2O→Acetoacetate+CoA (conversion 3.2)

(iii) Acetoacetate to Acetone and CO2 (conversion 4)

(iv) Acetone and NAD(P)H and H+ to Isopropanol and NAD(P)+ (conversion 5)

wherein at least one DNA molecule is heterologous to said microbial host cell and wherein said microbial host cell produces isopropanol.

In yet another embodiment, there is provided a method for the production of isopropanol comprising:

(a) providing a recombinant microbial host cell comprising each of the DNA molecules encoding a polypeptide or group of polypeptides that catalyze the conversion:

(i) Acetyl-CoA to Acetate and CoA (conversion 1)

(ii) Acetyl-CoA to Acetoacetyl-CoA and CoA (conversion 2)

(iii) Acetoacetyl-CoA and Acetate to Acetoacetate and Acetyl-CoA (conversion 3.1)

(iv) Acetoacetate to Acetone and CO2 (conversion 4)

(v) Acetone and NAD(P)H and H+ to Isopropanol and NAD(P)+ (conversion 5)

wherein the at least one DNA molecule is heterologous to said microbial host cell;
(b) contacting the host cell of (i) with a fermentable carbon substrate in a fermentation medium under conditions whereby isopropanol is produced; and
(c) recovering said isopropanol.

In still another embodiment, there is provided an isopropanol containing fermentation medium produced by a method comprising:

(a) providing a recombinant microbial host cell comprising each of the DNA molecules encoding a polypeptide or group of polypeptides that catalyze the conversion:

(i) Acetyl-CoA to Acetate and CoA (conversion 1)

(ii) Acetyl-CoA to Acetoacetyl-CoA and CoA (conversion 2)

(iii) Acetoacetyl-CoA and Acetate to Acetoacetate and Acetyl-CoA (conversion 3.1)

(iv) Acetoacetate to Acetone and CO2 (conversion 4)

(v) Acetone and NAD(P)H and H+ to Isopropanol and NAD(P)+ (conversion 5)

wherein at least one DNA molecule is heterologous to said microbial host cell and
(b) contacting the host cell of (i) with a fermentable carbon substrate in a fermentation medium under conditions whereby isopropanol is produced; and
(c) recovering said isopropanol.

In another embodiment, there is provided a method for the production of isopropanol comprising:

(a) providing a recombinant microbial host cell comprising each of the DNA molecules encoding a polypeptide that catalyzes the conversion:

(i) Acetyl-CoA to Acetoacetyl-CoA and CoA (conversion 2)

(ii) Acetoacetyl-CoA+H2O→Acetoacetate+CoA (conversion 3.2)

(iii) Acetoacetate to Acetone and CO2 (conversion 4)

(iv) Acetone and NAD(P)H and H+ to Isopropanol and NAD(P)+ (conversion 5)

wherein at least one DNA molecule is heterologous to said microbial host cell and
(b) contacting the host cell of (i) with a fermentable carbon substrate in a fermentation medium under conditions whereby isopropanol is produced.

In yet another embodiment, there is provided a method for the production of isopropanol comprising:

(a) providing a recombinant microbial host cell comprising each of the DNA molecules encoding a polypeptide that catalyzes the conversion:

(i) Acetyl-CoA to Acetoacetyl-CoA and CoA (conversion 2)

(ii) Acetoacetyl-CoA+H2O→Acetoacetate+CoA (conversion 3.2)

(iii) Acetoacetate to Acetone and CO2 (conversion 4)

(iv) Acetone and NAD(P)H and H+ to Isopropanol and NAD(P)+ (conversion 5)

wherein at least one DNA molecule is heterologous to said microbial host cell;
(b) contacting the host cell of (i) with a fermentable carbon substrate in a fermentation medium under conditions whereby isopropanol is produced; and
(c) recovering said isopropanol.

In still another embodiment, there is provided an isopropanol containing fermentation medium produced by a method comprising:

(a) providing a recombinant microbial host cell comprising each of the DNA molecules encoding a polypeptide that catalyzes the conversion:

(i) Acetyl-CoA to Acetoacetyl-CoA and CoA (conversion 2)

(ii) Acetoacetyl-CoA+H2O→Acetoacetate+CoA (conversion 3.2)

(iii) Acetoacetate to Acetone and CO2 (conversion 4)

(iv) Acetone and NAD(P)H and H+ to Isopropanol and NAD(P)+ (conversion 5)

wherein at least one DNA molecule is heterologous to said microbial host cell;
(b) contacting the host cell of (i) with a fermentable carbon substrate in a fermentation medium under conditions whereby isopropanol is produced; and
(c) recovering said isopropanol.
In another embodiment, there is provided

Isopropanol produced by a method comprising:

(a) providing recombinant microbial host cell comprising each of the DNA molecules encoding a polypeptide or group of polypeptides that catalyze the conversion:

(i) Acetyl-CoA to Acetate and CoA (conversion 1)

(ii) Acetyl-CoA to Acetoacetyl-CoA and CoA (conversion 2)

(iii) Acetoacetyl-CoA and Acetate to Acetoacetate and Acetyl-CoA (conversion 3.1)

(iv) Acetoacetate to Acetone and CO2 (conversion 4)

(v) Acetone and NAD(P)H and H+ to Isopropanol and NAD(P)+ (conversion 5)

wherein at least one DNA molecule is heterologous to said microbial host cell;
(b) contacting the host cell of (i) with a fermentable carbon substrate in a fermentation medium under conditions whereby isopropanol is produced; and
(c) recovering said isopropanol.

In yet another embodiment, there is provided isopropanol produced by a method comprising:

(a) providing a recombinant microbial host cell comprising each of the DNA molecules encoding a polypeptide that catalyzes the conversion:

(i) Acetyl-CoA to Acetoacetyl-CoA and CoA (conversion 2)

(ii) Acetoacetyl-CoA+H2O→Acetoacetate+CoA (conversion 3.2)

(iii) Acetoacetate to Acetone and CO2 (conversion 4)

(iv) Acetone and NAD(P)H and H+ to Isopropanol and NAD(P)+ (conversion 5)

wherein the at least one DNA molecule is heterologous to said microbial host cell;
(b) contacting the host cell of (i) with a fermentable carbon substrate in a fermentation medium under conditions whereby isopropanol is produced; and
(c) recovering said isopropanol.

Other embodiments are also disclosed.

### BRIEF DESCRIPTION OF THE DRAWINGS

FIG. 1 illustrates the metabolic pathways involved in the conversion of glucose to acids and solvents in Clostridium acetobutylicum (A). Other strains of the genus Clostridium produce isopropanol by reduction of acetone via an alcohol dehydrogenase (B).

FIGS. 2A and 2B illustrate a pathway in E. coli from glucose to isopropanol according to embodiments of the present disclosure. The pathway is shown under aerobic conditions (FIG. 2A) and anaerobic conditions (FIG. 2B).

FIG. 3 depicts plasmid pACT, also referred to herein as pGV1031, containing the thl, ctfA, ctfB, and adc genes from Clostridium acetobutylicum which are expressed from the native thiolase promoter.

FIG. 4 depicts plasmid pGV1093 containing the C. beijerinckii adhI open reading frame inserted between the EcoRI and BamHI sites in the pUC19 plasmid vector.

FIG. 5 depicts plasmid pGV1259 containing the C. beijerinckii adhI gene which is expressed from the P_{LlacO-1} promoter.

FIG. 6 depicts plasmid pGV1699 containing the C. acetobutylicum thl, ctfA, ctfB, and adc genes expressed from the native thl promoter as well as the C. beijerinckii adhI gene expressed form the P_{LlacO-1} promoter.

### BACKGROUND OF THE INVENTION

Microorganisms of the genus Clostridium have been reported to produce isopropanol, together with other solvents and acids, by fermentation. George et al. reported five species of Clostridia that produce isopropanol in addition to butanol or butanol and acetone (George H A, Johnson J L, Moore W E, Holdeman L V, Chen J S. Acetone, Isopropanol, and Butanol Production by Clostridium beijerinckii (syn. Clostridium butylicum) and Clostridium aurantibutyricum. Appl. Environ. Microbiol. 1983. 45(3):1160-1163). C. beijerinckii VPI2968 produced 9.8 mM isopropanol and 44.8 mM butanol. C. beijerinckii VPI2982 produced 1.6 mM isopropanol and 41.3 mM butanol. “C. butylicum” NRRL B593 produced 8.0 mM isopropanol and 61.7 mM butanol. C. aurantibutyricum ATCC 17777 produced 4.5 mM isopropanol, 45.4 mM butanol, and 20.5 mM acetone. C. aurantibutyricum NCIB 10659 produced 10.0 mM isopropanol, 42.4 mM butanol, and 14.5 mM acetone. Another report described strain 172CY that produces isopropanol and butanol in a continuous process using a CA-alginate immobilized fermenter (Araki K, Minami T, Sueki M, Kimura T. Continuous Fermentation by Butanol-Isopropanol Producing Microorganisms Immobilized by Ca-Alginate. J Soc Fermentation and Bioengineering. 1993. 71(1):9-14).

Bermejo et al. disclose the heterologous expression in E. coli of an “acetone operon” composed of four Clostridium acetobutylicum genes (Bermejo et al., Appl Environ Microbiol. 1998 March; 64(3):1079-85). Expression of this acetone pathway allowed the production of acetone from glucose in E. coli.

The four clostridial genes of the acetone pathway described by Bermejo encode three enzymes that can convert acetyl-coenzyme A (acetyl-CoA) and acetate into acetone. In the first step, the enzyme thiolase, which is encoded by the thl gene, generates acetoacetyl-CoA from two acetyl-CoA molecules by a condensation reaction. The enzyme acetoacetyl-CoA:acetate/butyrate:CoA transferase (CoAT), which is encoded by the ctfA and the ctfB genes, converts acetoacetyl-CoA and acetate into acetoacetate and acetyl-CoA. In the final step, acetoacetate decarboxylase (AADC), which is encoded by the adc gene, converts the acetoacetate into acetone and carbon dioxide.

Because C. acetobutylicum does not possess a secondary alcohol dehydrogenase, it is unable to produce the secondary alcohol isopropanol from the ketone substrate acetone. However, other species have been identified that contain either a primary-secondary alcohol dehydrogenase or a secondary alcohol dehydrogenase that are capable of converting acetone to isopropanol. For example, a primary-secondary alcohol dehydrogenase was characterized from two strains (NRRL B593 and NESTE 255) of Clostridium beijerinckii (Ismaiel, A. A., Zhu, C.-X., Colby, G. D. and Chen, J.-S. Purification and Characterization of a primary-secondary alcohol dehydrogenase from two strains of Clostridium beijerinckii. J. Bacteriol. 1993 175:5097-5105). This enzyme was shown to depend on NADPH and could convert both acetone and butyraldehyde to the corresponding alcohols isopropanol and n-butanol, respectively. Similarly, a secondary alcohol dehydrogenase from a strain (AIU 652) of Burkholderia sp. has been characterized (Isobe, K., and Wakao, N. Thermostable NAD+-dependent (R)-specific secondary alcohol dehydrogenase from cholesterol-utilizing Burkholderia sp. AIU 652. J. Biosci. Bioengr. 2003 96(4):387-393). This enzyme was shown to be NADH dependent.

The conversion of a carbon source into isopropanol production using heterologously expressed fermentative pathways, for example in E. coli, has not been reported.

Embodiments of the invention include recombinant microorganisms that contain a pathway to produce isopropanol and these microorganisms are used to produce isopropanol where at least one enzyme of the pathway is heterologous to the microorganism. Use of a heterologous host allows genomic manipulations to be performed quickly since a host can be chosen in having better understood molecular biology, and having far better developed molecular biology techniques, than that of the Clostridia species discussed above. Additionally, heterologous expression also avoids complications by native or endogenous regulation.

### DETAILED DESCRIPTION OF THE INVENTION

### Definitions

As used herein, the term “microorganism” includes prokaryotic and eukaryotic microbial species from the domains Archaea, Bacteria and Eukaryote, the latter including yeast and filamentous fungi, protozoa, algae, or higher Protista. The terms “cell,” “microbial cells,” and “microbes” are used interchangeably with the term microorganism.

“Gram-negative bacteria” include cocci, nonenteric rods and enteric rods. The genera of Gram-negative bacteria include, for example, Neisseria, Spirillum, Pasteurella, Brucella, Yersinia, Francisella, Haemophilus, Bordetella, Escherichia, Salmonella, Shigella, Klebsiella, Proteus, Pseudomonas, Bacteroides, Acetobacter, Aerobacter, Agrobacterium, Azotobacter, Myxococcus, Spirilla, Serratia, Vibrio, Rhizobium, Chlamydia, Rickettsia, Treponema and Fusobacterium.

“Gram positive bacteria” include cocci, nonsporulating rods and sporulating rods. The genera of gram positive bacteria include, for example, Actinomyces, Bacillus, Clostridium, Corynebacterium, Erysipelothrix, Lactobacillus, Listeria, Mycobacterium, Nocardia, Staphylococcus, Streptococcus and Streptomyces.

The term “carbon source” generally refers to a substrate or compound suitable to be used as a source of carbon for prokaryotic or simple eukaryotic cell growth. Carbon sources may be in various forms, including, but not limited to polymers, carbohydrates, acids, alcohols, aldehydes, ketones, amino acids, peptides, etc. These include, for example, various monosaccharides such as glucose, oligosaccharides, polysaccharides, cellulosic material, saturated or unsaturated fatty acids, succinate, lactate, acetate, ethanol, etc., or mixtures thereof. The carbon source may additionally be a product of photosynthesis, including, but not limited to glucose. The term “carbon source” may be used interchangeably with the term “energy source,” since in chemoorganotrophic metabolism the carbon source is used both as an electron donor during catabolism as well as a source of carbon during cell growth.

Carbon sources which serve as suitable starting materials for the production of isopropanol include, but are not limited to, biomass hydrolysates, glucose, starch, cellulose, hemicellulose, xylose, lignin, lignin compounds, dextrose, fructose, galactose, corn, liquefied corn meal, corn steep liquor (a byproduct of corn wet milling process that contains nutrients leached out of corn during soaking), molasses, lignocellulose, and maltose. Photosynthetic organisms can additionally produce a carbon source as a product of photosynthesis. In an embodiment, carbon sources may be selected from biomass hydrolysates and glucose. Glucose, dextrose and starch can be from an endogenous or exogenous source.

It should be noted that other carbon sources which may be more accessible, inexpensive, or both, can be substituted for glucose with relatively minor modifications to the host microorganisms. For example, in certain embodiments, use of other renewable and economically feasible substrates may be preferred. These may include agricultural waste, starch-based packaging materials, corn fiber hydrolysate, soy molasses, fruit processing industry waste, and whey permeate, etc.

As used herein, the term “yield” refers to the amount of product per amount of carbon source in g/g. The yield may be exemplified for glucose as the carbon source. It is understood unless otherwise noted that yield is expressed as a percentage of the theoretical yield. In reference to a microorganism or metabolic pathway, “theoretical yield” is defined as the maximum amount of product that can be generated per total amount of substrate as dictated by the stoichiometry of the metabolic pathway used to make the product. For example, the theoretical yield for one typical conversion of glucose to isopropanol is 0.33 g/g. As such, a yield of isopropanol from glucose of 29.7 g/g would be expressed as 90% of theoretical or 90% theoretical yield. It is understood that while in the present disclosure the yield is exemplified for glucose as a carbon source, the invention can be applied to other carbon sources and the yield may vary depending on the carbon source used. One skilled in the art can calculate yields on various carbon sources.

The microorganisms herein disclosed are, in some cases, engineered using genetic engineering techniques, to provide microorganisms which utilize heterologously expressed enzymes to produce isopropanol at high yield.

The term “enzyme” as used herein refers to any substance that catalyzes or promotes one or more chemical or biochemical reactions, which usually includes enzymes totally or partially composed of a polypeptide, but can include enzymes composed of a different molecule including polynucleotides.

The term “polynucleotide” is used herein interchangeably with the term “nucleic acid” and refers to an organic polymer composed of two or more monomers including nucleotides, or nucleosides, including but not limited to single stranded or double stranded, sense or antisense deoxyribonucleic acid (DNA) of any length and, where appropriate, single stranded or double stranded, sense or antisense ribonucleic acid (RNA) of any length, including siRNA. The term “nucleotide” refers to any of several compounds that consist of a ribose or deoxyribose sugar joined to a purine or a pyrimidine base and to a phosphate group, and that are the basic structural units of nucleic acids. The term “nucleoside” refers to a compound (as guanosine or adenosine) that consists of a purine or pyrimidine base combined with deoxyribose or ribose and is found especially in nucleic acids. Accordingly, the term polynucleotide includes nucleic acids of any length, DNA, RNA, analogs and fragments thereof. A polynucleotide of three or more nucleotides is also called “nucleotidic oligomer” or “oligonucleotide”.

The term “protein” or “polypeptide” as used herein indicates an organic polymer composed of two or more amino acidic monomers and/or analogs thereof. As used herein, the term “amino acid” or “amino acidic monomer” refers to any natural and/or synthetic amino acids including glycine and both D or L optical isomers. Accordingly, the term polypeptide includes amino acidic polymer of any length including full length proteins, and peptides as well as analogs and fragments thereof.

As used herein, the term “pathway” refers to a biological process including one or more enzymatically controlled chemical reactions by which a substrate is converted into a product. Accordingly, a pathway for the conversion of a carbon source to isopropanol is a biological process including one or more enzymatically controlled reactions by which the carbon source is converted into isopropanol. A “heterologous pathway” refers to a pathway wherein at least one of the one or more chemical reactions is catalyzed by at least one heterologous enzyme. On the other hand, a “native pathway” refers to a pathway wherein the one or more chemical reactions are catalyzed by a native enzyme.

The term “heterologous” or “exogenous” as used herein with reference to enzymes and polynucleotides, indicates enzymes or polynucleotides that are expressed in an organism other than the organism from which they originated or are found in nature, independently on the level of expression that can be lower, equal to, or higher than the level of expression of the molecule in the native microorganism.

On the other hand, the term “native” or “endogenous” as used herein with reference to enzymes and polynucleotides, indicates enzymes and polynucleotides that are expressed hi the organism in which they originated or are found in nature, independently of the level of expression that can be lower equal or higher than the level of expression of the molecule in the native microorganism

The terms “host” or “host cells” are used interchangeably herein and refer to microorganisms, native or wild-type, eukaryotic or prokaryotic that can be engineered for the conversion of a carbon source to isopropanol. The terms “host” and “host cells” refers not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The terms “activate” or “activation” as used herein with reference to a biologically active molecule, such as an enzyme, indicates any modification in the genome and/or proteome of a microorganism that increases the biological activity of the biologically active molecule in the microorganism. Exemplary activations include but are not limited to modifications that result in the conversion of the molecule from a biologically inactive form to a biologically active form and from a biologically active form to a biologically more active form, and modifications that result in the expression of the biologically active molecule in a microorganism wherein the biologically active molecule was previously not expressed or expressed at lower concentrations. For example, activation of a biologically active molecule can be performed by expressing a native or heterologous polynucleotide encoding for the biologically active molecule in the microorganism, by expressing a native or heterologous polynucleotide encoding for an enzyme involved in the pathway for the synthesis of the biological active molecule in the microorganism, or by expressing a native or heterologous molecule that enhances the expression of the biologically active molecule in the microorganism.

In particular, the recombinant microorganisms herein disclosed are engineered to activate, and, in particular, express heterologous enzymes that can be used in the production of isopropanol. In particular, in certain embodiments, the recombinant microorganisms are engineered to activate heterologous enzymes that catalyze the conversion of acetyl-CoA to isopropanol.

As used herein, “deleting genes” means that a gene is deleted or otherwise mutated to inactivate the gene. Deletions can be of coding sequences or regulatory sequences provided they do not tend to revert and provided they inactivate the gene product (or gene products as the case may be). Operons can be inactivated as well.

As used herein, “sequence identity” refers to the occurrence of exactly the same nucleotide or amino acid in the same position in aligned sequences. “Sequence similarity” takes approximate matches into account, and is meaningful only when such substitutions are scored according to some measure of “difference” or “sameness” with conservative or highly probable substitutions assigned more favorable scores than non-conservative or unlikely ones.

In certain embodiments, any enzyme that catalyzes a conversion described in herein may be used.

In certain embodiments, any homologous enzymes that are at least about 70%, 80%, 90%, 95%, 99% identical, or sharing at least about 60%, 70%, 80%, 90%, 95% sequence identity to any of the enzymes of the isopropanol pathway may be used in place of the enzymes described. These enzymes sharing the requisite sequence identity or similarity may be wild-type enzymes from a different organism, or may be artificial, i.e., recombinant, enzymes.

In certain embodiments, any genes encoding for enzymes with the same activity as any of the enzymes of the isopropanol pathway may be used in place of the enzymes. These enzymes may be wild-type enzymes from a different organism, or may be artificial, recombinant or engineered enzymes.

Additionally, due to the inherent degeneracy of the genetic code, other nucleic acid sequences which encode substantially the same or a functionally equivalent amino acid sequence can also be used express the polynucleotide encoding such enzymes. As will be understood by those of skill in the art, it can be advantageous to modify a coding sequence to enhance its expression in a particular host. The codons that are utilized most often in a species are called “optimal codons”, and those not utilized very often are classified as “rare or low-usage codons”. Codons can be substituted to reflect the preferred codon usage of the host, a process sometimes called “codon optimization” or “controlling for species codon bias.” Methodology for optimizing a nucleotide sequence for expression in a plant is provided, for example, in U.S. Pat. No. 6,015,891.

Expression of the genes may be accomplished by conventional molecular biology techniques. For example, the heterologous genes can be under the control of an inducible promoter or a constitutive promoter. The heterologous genes may either be integrated into a chromosome of the host microorganism, or exist as an extra-chromosomal genetic elements that can be stably passed on (“inherited”) to daughter cells. Such extra-chromosomal genetic elements (such as plasmids, BAC, YAC, etc.) may additionally contain selection markers that ensure the presence of such genetic elements in daughter cells.

Methods of over-expressing, expressing at various levels, and repressing expression of genes in microorganisms are well known in the art, and any such method is contemplated for use in the construction of the microorganisms of the present invention. For example, integrational mutagenesis is a genetic engineering technique that can be used to selectively inactivate undesired genes from a host chromosome. Pursuant to this technique, a fragment of a target gene is cloned into a non-replicative vector with a selection marker to produce a non-replicative integrational plasmid. The partial gene in the non-replicative plasmid can be recombined with the internal homologous region of the original target gene in the parental chromosome, which results in insertional inactivation of the target gene.

Any method can be used to introduce an exogenous nucleic acid molecule into microorganisms and many such methods are well known to those skilled in the art. For example, transformation, electroporation, conjugation, and fusion of protoplasts are common methods for introducing nucleic acid into microorganisms.

The exogenous nucleic acid molecule contained within a microorganism described herein can be maintained within that cell in any form. For example, exogenous nucleic acid molecules can be integrated into the genome of the cell or maintained in an episomal state that can stably be passed on (“inherited”) to daughter cells. Such extra-chromosomal genetic elements (such as plasmids, etc.) may additionally contain selection markers that ensure the presence of such genetic elements in daughter cells. Moreover, the microorganisms can be stably or transiently transformed. In addition, the microorganisms described herein can contain a single copy, or multiple copies of a particular exogenous nucleic acid molecule as described above.

Methods for expressing polypeptide from an exogenous nucleic acid molecule are well known to those skilled in the art. Such methods include, without limitation, constructing a nucleic acid such that a regulatory element promotes the expression of a nucleic acid sequence that encodes the desired polypeptide. Typically, regulatory elements are DNA sequences that regulate the expression of other DNA sequences at the level of transcription. Thus, regulatory elements include, without limitation, promoters, enhancers, and the like. For example, the exogenous genes can be under the control of an inducible promoter or a constitutive promoter.

Moreover, methods for expressing a polypeptide from an exogenous nucleic acid molecule in microorganisms are well known to those skilled in the art. In another embodiment, heterologous control elements can be used to activate or repress expression of endogenous genes. Additionally, when expression is to be repressed or eliminated, the gene for the relevant enzyme, protein or RNA can be eliminated by known deletion techniques.

As described herein, microorganisms within the scope of the disclosure can be identified by techniques specific to the particular enzyme being expressed, over-expressed or repressed. Methods of identifying the strains with the desired phenotype are well known to those skilled in the art. Such methods include, without limitation, PCR and nucleic acid hybridization techniques such as northern and Southern blot analysis, altered growth capabilities on a particular substrate or in the presence of a particular substrate, a chemical compound, a selection agent and the like. In some cases, immunohistochemistry and biochemical techniques can be used to determine if a cell contains a particular nucleic acid by detecting the expression of the encoded polypeptide. For example, an antibody having specificity for an encoded enzyme can be used to determine whether or not a particular cell contains that encoded enzyme. Further, biochemical techniques can be used to determine if a cell contains a particular nucleic acid molecule encoding an enzymatic polypeptide by detecting a product produced as a result of the expression of the enzymatic polypeptide. For example, transforming a cell with a vector encoding an alcohol dehydrogenase (ADH) and detecting isopropanol in the cytosol, cell extracts or culture medium supernatant resulting from the ADH catalyzed conversion of acetone to isopropanol indicates that the vector is both present and the gene product is active. Methods for detecting specific enzymatic activities or the presence of particular products are well known to those skilled in the art.

Metabolization of a carbon source is said to be “balanced” when the NAD(P)H produced during the oxidation reactions of the carbon source equals the NAD(P)H utilized to convert the carbon source to metabolization end products. Under these conditions, all the NAD(P)H is recycled. Without recycling, the NAD(P)H/NAD(P)⁺ ratio becomes imbalanced and will cause the organism to ultimately die unless alternate metabolic pathways are available to maintain a balanced NAD(P)H/NAD(P)⁺ ratio.

In an embodiment, the recombinant microorganisms is capable of converting a carbon source to isopropanol.

In certain embodiments, the recombinant microorganism of the present disclosure is capable of converting a carbon source to acetyl-CoA and of converting acetyl-CoA to isopropanol.

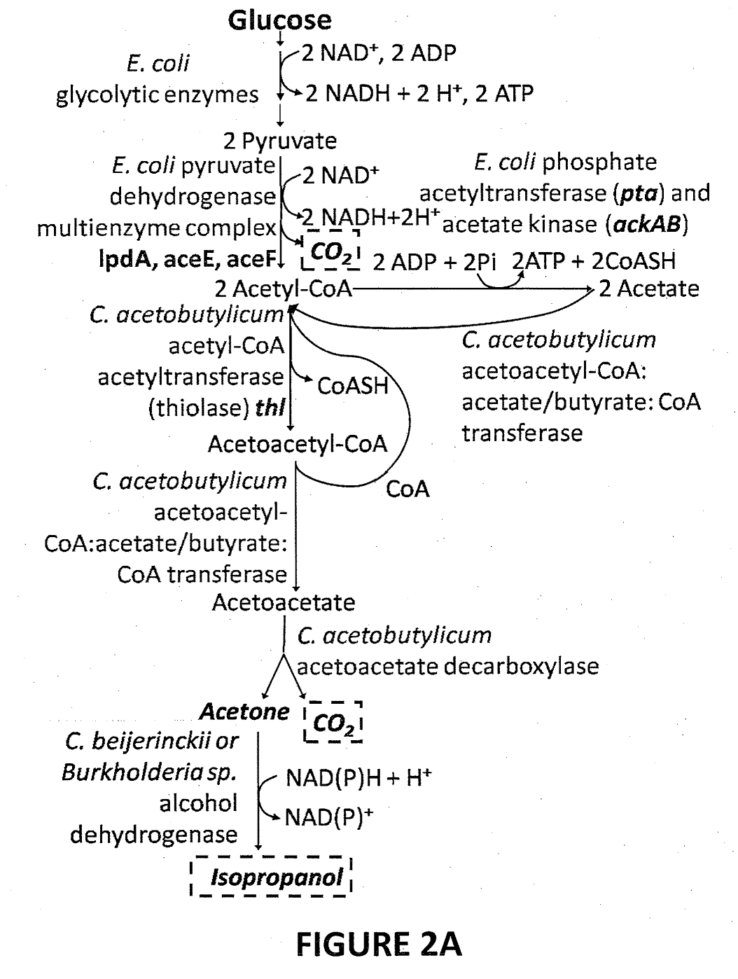

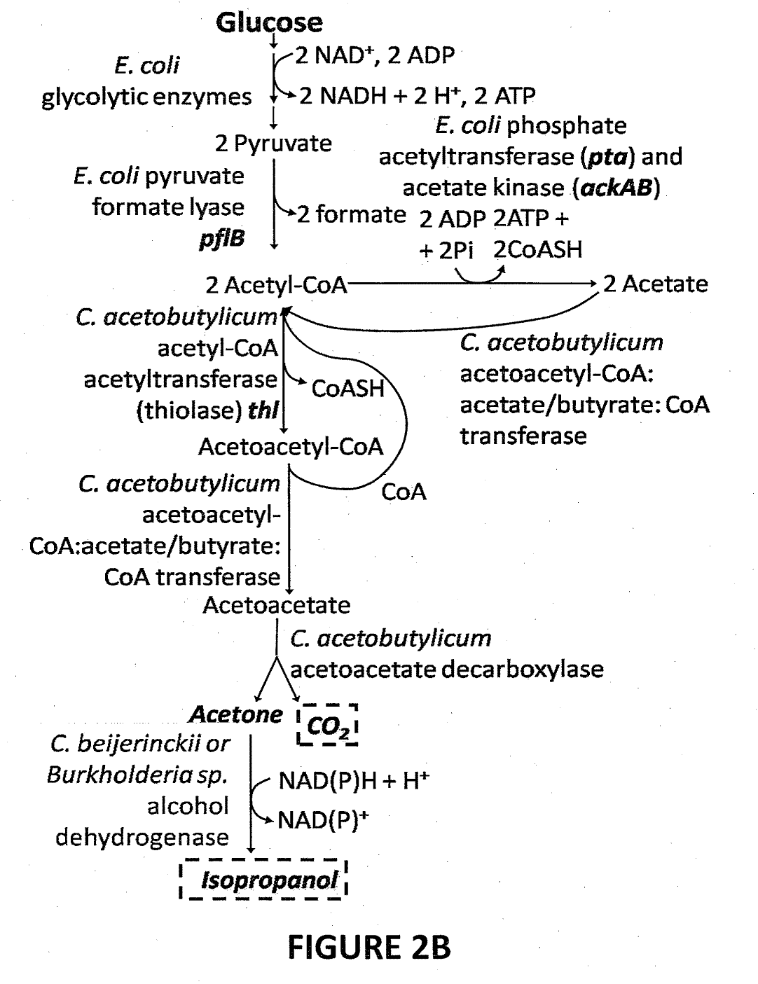

Host organisms can be engineered to express a metabolic pathway for the conversion of acetyl-CoA to isopropanol wherein at least one of the pathway enzymes is heterologous to the host (FIGS. 2A and 2B).

In certain embodiments, the recombinant microorganism of the present disclosure is capable of catalyzing the following chemical conversions (Pathway 1):

Acetyl-CoA→Acetate+CoA (conversion 1)

2Acetyl-CoA→Acetoacetyl-CoA+CoA (conversion 2)

Acetoacetyl-CoA+Acetate→Acetoacetate+Acetyl-CoA (conversion 3.1)

Acetoacetate→Acetone+CO₂ (conversion 4)

Acetone+NAD(P)H+H⁺→Isopropanol+NAD(P)⁺ (conversion 5)

Where the net reaction is as follows:

2Acetyl-CoA+NAD(P)H+H⁺→Isopropanol+NAD(P)⁺+CO₂+2CoA

and where the theoretical is 1 mole of isopropanol per mole of glucose or 0.33 gram isopropanol per gram of glucose.

In certain embodiments, the recombinant microorganism of the present disclosure expresses genes encoding the following enzymes that catalyze conversions 1, 2, 3.1, 4 and 5 of

Pathway 1:

phosphate acetyltrasferase and acetate kinase (catalyzes conversion 1)

acetyl-CoA-acetyltransferase(thiolase) (catalyzes conversion 2)

acetoacetyl-CoA:acetate/butyrate coenzyme-A transferase (catalyzes conversion 3.1)

acetoacetate decarboxylase (catalyzes conversion 4)

secondary alcohol dehydrogenase (catalyzes conversion 5)

In certain embodiments, the recombinant microorganism of the present disclosure is capable of catalysing the following chemical conversions (Pathway 2):

2Acetyl-CoA→Acetoacetyl-CoA+CoA (conversion 2)

Acetoacetyl-CoA+H₂O→Acetoacetate+CoA (conversion 3.2)

Acetoacetate→Acetone+CO₂ (conversion 4)

Acetone+NAD(P)H+H⁺→Isopropanol+NAD(P)⁺ (conversion 5)

Where the net reaction is as follows:

2Acetyl-CoA+NAD(P)H+H⁺+H₂O→Isopropanol+NAD(P)⁺+CO₂+2CoA

and where the theoretical is 1 mole of isopropanol per mole of glucose or 0.33 g isopropanol per gram of glucose.

In certain embodiments, the recombinant microorganism of the present disclosure expresses genes encoding the following enzymes that catalyze above reactions 2, 3.2, 4, and 5 of Pathway 2:

acetyl-CoA-acetyltransferase(thiolase) (catalyzes conversion 2)

acetoacetyl-CoA hydrolase (catalyzes conversion 3.2)

acetoacetate decarboxylase (catalyzes conversion 4)

secondary alcohol dehydrogenase (catalyzes conversion 5)

In certain embodiments, at least one of the genes expressed within the recombinant microorganism is heterologous to the microorganism. Such heterologous genes may be identified within and obtained from a heterologous microorganism (such as Clostridium acetobutylicum or Clostridium beijerinckii), and can be introduced into an appropriate host using conventional molecular biology techniques. The at least one of heterologous genes enable the recombinant microorganism to produce isopropanol or a metabolic intermediate thereof, at least in an amount greater than that produced by the wild-type counterpart microorganism.

Useful microorganisms that can be used as recombinant hosts may be either eukaryotic or prokaryotic microorganisms. While Escherichia is one of the hosts that may be used according to the present disclosure, other hosts may be used, including yeast strains such as Saccharomyces strains.

In certain embodiments, other suitable recombinant hosts include, but are not limited to, Pichia, Hansenula, Yarrowia, Aspergillus, Kluyveromyces, Pachysolen, Rhodotorula, Zygosaccharomyces, Galactomyces, Schizosaccharomyces, Torulaspora, Debaryomyces, Williopsis, Dekkera, Kloeckera, Metschnikowia and Candida.

In certain embodiments the recombinant hosts include, but are not limited to, Arthrobacter, Bacillus, Brevibacterium, Clostridium, Corynebacterium, Gluconobacter, Nocardia, Pseudomonas, Rhodococcus, Salmonella, Streptomyces, and Xanthomonas.

In certain embodiments, such hosts include E. coli W3110, E. coli B, Pseudomonas oleovorans, Pseudomonas fluorescens, Pseudomonas putida, and Saccharomyces cerevisiae.

In one embodiment, the engineered microorganism is an E. coli.

In another embodiment, the engineered microorganism is yeast, for example Saccharomyces cerevisiae. Yeasts have pathways in both the cytosol and the mitochondria that generate acetyl-CoA. Because the conversion in yeast of acetyl-CoA to isopropanol takes place in the cytosol, it is desirable for recombinant yeast of the present invention to have increased cytosolic concentrations of acetyl-CoA relative to wild-type levels. Additionally, mitochondrial concentrations of acetyl-CoA can be reduced.

In certain embodiments conversion 1 is catalyzed by enzymes classified as E.C. 2.3.1.8 and E.C. 2.7.2.1 that convert acetyl-CoA to acetate via the intermediate acetylphosphate, e.g., the enzymes phosphate acetyltrasferase (pta) and acetate kinase (ackAB) from either E. coli or Clostridium species. Conversion 2 is catalyzed by an enzyme classified as E.C. 2.3.1.19, i.e., an cetyl-CoA acetyltransferase (thiolase). Conversion 3.1 is catalyzed by an enzyme classified as E.C. 2.8.3.9, i.e., an acetoacetyl-CoA:acetate/butyrate coenzyme-A transferase (CoAT). Conversion 3.2 is catalyzed by an enzyme classified as EC 3.1.2.11, i.e., an acetoacetyl-CoA hydrolase. Conversion 4 is catalyzed by an enzyme classified as E.C. 4.1.1.4, i.e., an acetoacetate decarboxylase. Conversion 5 is catalyzed by an alcohol dehydrogenase, such as an alcohol dehydrogenase from the C. beijerinckii, the Burkholderia sp., or Thermoanaerobacter brockii.

In one embodiment, a recombinant microorganism provided herein includes activation of enzymes that convert acetyl-CoA to acetate via the intermediate acetylphosphate. In one embodiment, activation results from the expression of the endogenous enzymes. In another embodiment, activation results from the expression of heterologous enzymes. Suitable enzymes, include, but are not limited to, phosphate acetyltrasferase, which catalyzes the conversion of acetyl-CoA to acetylphosphate, and acetate kinase, which catalyzes the conversion of acetylphosphate to acetate. In one embodiment, these enzymes are encoded by pta and ackAB from E. coli or a Clostridium species.

In one embodiment, a recombinant microorganism provided herein is engineered to activate an acetyl-CoA acetyltransferase (thiolase) as compared to a parental microorganism. Thiolase (E.C. 2.3.1.19) catalyzes the condensation of an acetyl group onto an acetyl-CoA molecule. This enzyme has been overexpressed, amongst other enzymes, in E. coli under its native promoter for the production of acetone (Bermejo et al., Appl. Environ. Mirobiol. 64: 1079-1085, 1998).

In one embodiment, the increased thiolase expression results from the activation of an endogenous thiolase. In another embodiment, the increased thiolase expression results from the expression of a heterologous thiolase gene. In a further embodiment, the heterologous thiolase gene is from a Clostridium species. In yet a further embodiment, the thiolase is the C. acetobutylicum enzyme encoded by the gene thl (GenBank accession U08465, protein ID AAA82724.1), and whose amino acid sequence is given in SEQ ID NO: 4.

Other homologous thiolases include, but are not limited to, those from: C. pasteurianum (e.g., protein ID ABA18857.1), C. beijerinckii sp. (e.g., protein ID EAP59904.1 or EAP59331.1), Clostridium perfringens sp. (e.g., protein ID ABG86544.1, ABG83108.1), Clostridium difficile sp. (e.g., protein ID CAJ67900.1 or ZP_{—}01231975.1), Thermoanaerobacterium thermosaccharolyticum (e.g., protein ID CAB07500.1), Thermoanaerobacter tengcongensis (e.g., AAM23825.1), Carboxydothermus hydrogenoformans (e.g., protein ID ABB13995.1), Desulfotomaculum reducens MI-1 (e.g., protein ID EAR45123.1), Candida tropicalis (e.g., protein ID BAA02716.1 or BAA02715.1), Saccharomyces cerevisiae (e.g., protein ID AAA62378.1 or CAA30788.1), Bacillus sp., Megasphaera elsdenii, or Butryivibrio fibrisolvens, etc. In addition, an E. coli thiolase could also be active in a heterologously expressed isopropanol pathway. E. coli synthesizes two distinct 3-ketoacyl-CoA thiolases. One is a product of the fadA gene, the second is the product of the atoB gene.

In one embodiment, a recombinant microorganism provided herein is engineered to activate an acetoacetyl-CoA:acetate/butyrate coenzyme-A transferase (CoAT) as compared to a parental microorganism. CoAT (E.C. 2.8.3.9) transfers the coenzyme A from acetoacetyl-CoA to acetate resulting in the products acetoacetate and acetyl-CoA.

In one embodiment, the increased CoAT expression results from the activation of an endogenous CoAT. In another embodiment, the increased CoAT expression results from the expression of a heterologous CoAT gene. In a further embodiment, the heterologous CoAT gene is from a Clostridium species. In yet a further embodiment, the CoAT is the C. acetobutylicum enzyme encoded by the two genes ctfA (GenBank accession NC_{—}001988, protein ID NP_{—}149326.1) and ctfB (GenBank accession NC_{—}001988, protein ID NP_{—}149327.1), and whose amino acid sequences are given in SEQ ID NO:5 and SEQ ID NO:6, respectively.

In one embodiment, a recombinant microorganism provided herein is engineered to activate an acetoacetyl-CoA hydrolase as compared to a parental microorganism. Acetoacetyl-CoA hydrolase (EC 3.1.2.11) catalyzes the hydrolysis of acetoacetyl-CoA to form acetoacetate and CoA.

In one embodiment, the increased acetoacetyl-CoA hydrolase expression results from activation of an endogenous acetoacetyl-CoA hydrolase. In another embodiment, the increased acetoacetyl-CoA hydrolase expression results from the expression of a heterologous acetoacetyl-CoA hydrolase gene.

Suitable acetoacetyl-CoA hydrolases have been identified in mammalian cells (see e.g., Drummond, 1960; Baird, 1970; Baird, 1969; Zammit, 1979; Rous, 1976; Aragon, 1983; Patel, 1978; Patel, 1978). Alternatively, the substrate specificity of an acetyl-CoA hydrolase (E.C. 3.1.2.1) can be altered by protein engineering techniques such as ‘directed evolution’ so that it can convert acetoacetyl-CoA as a substrate. For example, the acetyl-CoA hydrolase Ach1p from Saccharomyces cerevisae (Genbank accession NP_{—}009538.1) can be used for this purpose.

In one embodiment, a recombinant microorganism provided herein is engineered to activate an acetoacetate decarboxylase as compared to a parental microorganism. Acetoacetate decarboxylase (E.C. 4.1.1.4) converts acetoacetate into acetone and carbon dioxide.

In one embodiment, the increased acetoacetate decarboxylase expression results from activation of an endogenous acetoacetate decarboxylase. In another embodiment, the increased acetoacetate decarboxylase expression results from the expression of a heterologous acetoacetate decarboxylase gene. In a further embodiment, the heterologous acetoacetate decarboxylase gene is from a Clostridium species. In yet a further embodiment, the acetoacetate decarboxylase is the C. acetobutylicum enzyme encoded by the adc gene (GenBank accession NC_{—}001988, protein ID NP_{—}149328.1), and whose amino acid sequence is given in SEQ ID NO: 7.

In one embodiment, a recombinant microorganism provided herein is engineered to activate an alcohol dehydrogenase (ADH) as compared to a parental microorganism. ADH reduces acetone to isopropanol with the oxidation of NAD(P)H to NAD(P)⁺.

In one embodiment, the increased ADH expression results from activation of an endogenous ADH. In another embodiment, the increased ADH expression results from the expression of a heterologous ADH gene. In a further embodiment, the heterologous ADH gene is from a Clostridium species. In yet a further embodiment, the ADH is the NADPH-dependant C. beijerinckii enzyme encoded by the adhI gene (GenBank accession AF157307, protein ID AAA23199.2), and whose amino acid sequence is given in SEQ ID NO: 8. Other suitable alcohol dehydrogenases, include, but are not limited to, the Burkholderia sp. AIU 652 enzyme, which is NADH-dependent or the Thermoanaerobacter brockii alcohol dehydrogenase (Genbank protein ID CAA46053.1) encoded by tbad gene (Genbank accession number X64841).

In certain embodiments, any enzyme that catalyzes the above described conversions may be used.

In certain embodiments, any homologous enzymes that are at least about 70%, 80%, 90%, 95%, 99% identical with respect to their amino acid sequence, or sharing at least about 60%, 70%, 80%, 90%, 95% sequence homology with respect to their amino acid sequence to any of the polypeptides described herein, can be used in place of these wild-type polypeptides. One skilled in the art can easily identify corresponding, homologous genes in other microorganisms by convention molecular biology techniques (such as sequence homology search, cloning based on homologous sequences, etc.).

Nucleic acid sequences that encode enzymes useful for generating metabolic intermediates of the isopropanol pathway disclosed herein (e.g., thiolase, phosphate acetyltrasferase, acetate kinase, acetoacetyl-CoA:acetate/butyrate coenzyme-A transferase, acetoacetate decarboxylase, acetoacetyl-CoA hydrolase, alcohol dehydrogenase) including homologs, variants, fragments, related fusion proteins, or functional equivalents thereof, are used in recombinant nucleic acid molecules that direct the expression of such polypeptides in appropriate host cells, such as bacterial or yeast cells. It is understood that the addition of sequences which do not alter the encoded activity of a nucleic acid molecule, such as the addition of a non-functional or non-coding sequence, is a conservative variation of the basic nucleic acid.

In one embodiment, all five genes encoding for enzymes that catalyze conversions of Pathway 1, namely conversions 1, 2, 3.1, 4, and 5 are expressed from a single plasmid. In this embodiment, several combinations are possible, including, but not limited to; all genes expressed on a high-copy, medium-copy, or low-copy plasmid; all genes expressed from a single promoter; all genes expressed each with their own promoter; and synthetic operons of one, two, three, and/or four genes expressed from several promoters. Methods for optimizing the expression level ratios of the genes to achieve high productivity are known to those skilled in the art and can be applied to the expression system for expression of these genes.

Further, in one embodiment, all five genes adhI, thl, ctfA, ctfB, and adc, are expressed from a single plasmid. In this embodiment, several combinations are possible, including, but not limited to; all genes expressed on a high-copy, medium-copy, or low-copy plasmid; all genes expressed from a single promoter; all genes expressed each with their own promoter; and synthetic operons of one, two, three, and/or four genes expressed from several promoters.

In one embodiment, all four genes encoding for enzymes that catalyze conversions of Pathway 2, namely conversions 2, 3.2, 4, and 5 are expressed from a single plasmid. In this embodiment, several combinations are possible, including but not limited to; all genes expressed on a high-copy, medium-copy, or low-copy plasmid; all genes expressed from a single promoter; all genes expressed each with their own promoter; and synthetic operons of one, two, three, and/or four genes expressed from several promoters. Methods for optimizing the expression level ratios of the genes to achieve high productivity are known to those skilled in the art and can be applied to the expression system for expression of these genes.

Many heterogeneously-expressed enzymes may not be initially optimized for use as a metabolic enzyme inside a host microorganism. However, these enzymes can usually be improved using protein engineering techniques, including directed evolution. In other words, even if the activity of an isopropanol-producing strain is low initially, it is possible to improve upon this pathway. For example, in directed evolution, genetic diversity is created by mutagenesis and/or recombination of one or more parental gene sequences. These altered genes are cloned back into a plasmid for expression in a suitable host organism (bacteria or yeast). Clones expressing improved enzymes are identified in a high-throughput screen, or in some cases, by selection, and the gene(s) encoding those improved enzymes are isolated and the process is applied iteratively until an enzyme with the desired activity is obtained. For example, using engineered E. coli strains, which contain the most effective variant of a desired isopropanol-producing pathway, directed evolution of the enzyme can be performed to obtain improved enzymes resulting in an improved isopropanol production pathway. Similar processes can also be used to identify and isolate strains with a higher isopropanol yield per glucose metabolized.

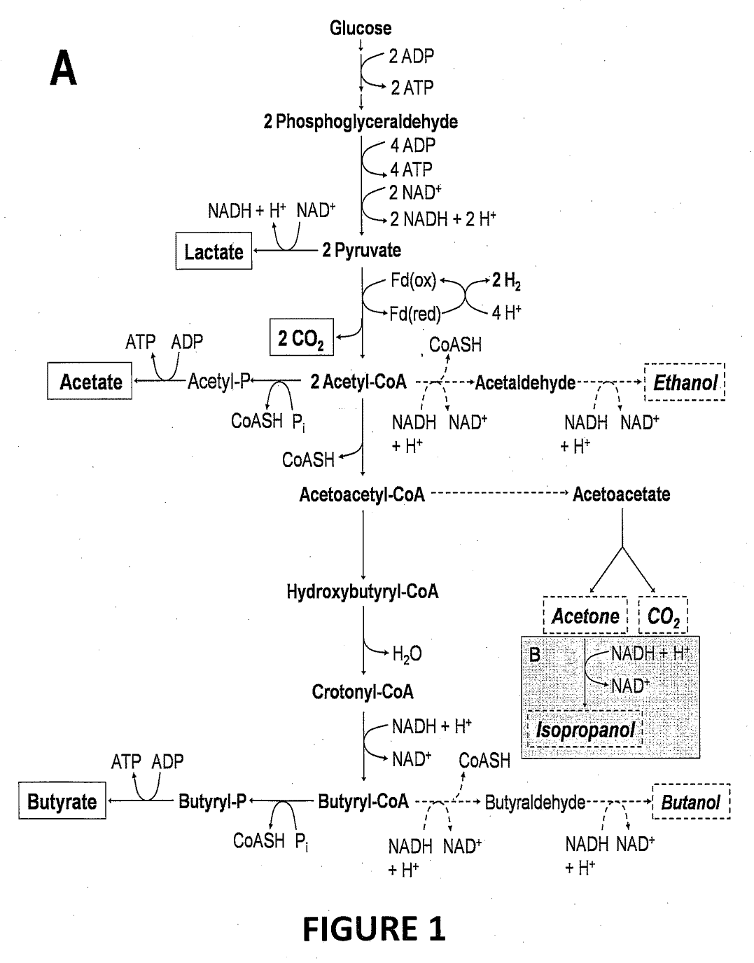

The production of isopropanol from a carbohydrate source by the metabolic pathways 1 and 2 is not balanced with respect to NAD(P)H produced and NAD(P)H consumed. For example, under anaerobic conditions in E. coli, the conversion of glucose to acetyl-CoA generates 2 moles of NAD(P)H, while the conversion of acetyl-CoA to isopropanol only requires 1 mole of NAD(P)H (see FIG. 1). Similarly under aerobic conditions in E. coli, the conversion of glucose to acetyl-CoA generates 4 moles of NAD(P)H while the conversion of acetyl-CoA to isopropanol requires 1 mole of NAD(P)H. Unless alternate metabolic pathways recycle NAD(P)H, the NAD(P)H/NAD(P)⁺ ratio will become imbalanced and will cause the organism to ultimately die.

In another embodiment, NADH that is not oxidized during the conversion of acetyl-CoA to isopropanol is otherwise oxidized so that metabolism is balanced with respect to NAD⁺ reduction and NADH oxidation.

In one embodiment, excess NADH is oxidized by native enzymes or metabolic pathways.

In another embodiment, excess NADH is oxidized by heterologously expressed enzymes or metabolic pathways.

In another embodiment, excess NAD(P)H produced during the conversion of a carbon source to isopropanol can be removed by coupling the oxidation of NAD(P)H to the reduction of a metabolic intermediate.

In yet another embodiment, such a metabolic intermediate is pyruvate or acetyl-CoA.

One solution is for the engineered isopropanol pathway to run under aerobic or microaerobic conditions, in which case excess reducing equivalents would be consumed by the native respiratory pathway(s) of the microorganisms. The overall net reactions for the production process from glucose to isopropanol under such conditions is as follows:

1Glucose+1.5O₂→1Isopropanol+3CO₂

It is preferable to divert as much carbon flux as possible to acetyl-CoA as substrate for the engineered isopropanol pathway. Competing pathways, such as the tricarboxylic acid (TCA) cycle under aerobic conditions, that consume acetyl-CoA should preferably be eliminated or impaired. The TCA cycle can be disrupted at the succinate dehydrogenase/fumarate reductase step or at the alpha-keto glutarate dehydrogenase step to prevent consumption of acetyl-CoA through this pathway and the consequent loss of carbon as CO₂. However, disruption of the TCA cycle must occur in such a way that all required anapleurotic pathways are maintained. It has been shown that flux through the TCA cycle can be decreased using either a succinate dehydrogenase/malate dehydrogenase double knockout (Fischer, E. and Sauer, U., Metabolic flux profiling of Escherichia coli mutants in central carbon metabolism using GC-MS, Eur. J. Biochem. 270:880-891 (2003)) or an alpha-ketoglutarate dehydrogenase mutant (Causey T. B., Zhou S., Shanmugam K. T., Ingram L. O., Engineering the metabolism of Escherichia coli W3110 for the conversion of sugar to redox-neutral and oxidized products: homoacetate production, Proc. Natl. Acad. Sci. USA. 100(3):825-32 (2003)).

Another solution that allows the engineered isopropanol pathway to operate anaerobically is to couple the isopropanol pathway with expression of another biocatalyst, such as a cytochrome P450 or a reductase, thereby consuming the remaining reducing equivalents to generate a redox-balanced pathway. One non-limiting example of this embodiment is to use an engineered P450 to convert propane to propanol while consuming reducing equivalents.

Alternatively, excess NAD(P)H produced during the conversion of a carbon source to isopropanol can be removed by a heterologously overexpressed hydrogenase, which couples the oxidation of NADH to the formation of hydrogen.

In certain embodiments of the current disclosure where the alcohol dehydrogenase is NADPH-dependent, endogenous processes that produce NADPH are upregulated. Examples of such processes include, but are not limited to, upregulating the pentose phosphate pathway and the activity of transhydrogenase enzymes.

In certain embodiments of the current disclosure where the alcohol dehydrogenase is NADPH-dependent, protein engineering techniques may be used to convert said NADPH-dependent alcohol dehydrogenase(s) to an NADH-dependent alcohol dehydrogenase(s).

In certain embodiments the second biochemical process comprises of culturing a recombinant microorganism of the invention in a suitable culture medium under suitable culture conditions.

Suitable culture conditions depend on the temperature optimum, pH optimum, and nutrient requirements of the host microorganism and are known by those skilled in the art. These culture conditions may be controlled by methods known by those skilled in the art.

For example, E. coli cells are typically grown at temperatures of about 25° C. to about 40° C. and a pH of about pH 4.0 to pH 8.0. Growth media used to produce isopropanol according to the present invention include common media such as Luria Bertani (LB) broth, EZ-Rich medium, and commercially relevant minimal media that utilize cheap sources of nitrogen, sulfur, phosphorus, mineral salts, trace elements and a carbon source as defined.

In certain embodiments the fermentation is performed using a batch reactor. In other embodiments, the fermentation can be done by fed-batch or continuos reactors. Fermentations may be performed under aerobic or anaerobic conditions, where anaerobic or microaerobic conditions are preferred during the isopropanol production phase.

The amount of isopropanol produced in the fermentation medium can be determined using a number of methods known in the art, for example, high performance liquid chromatography or gas chromatography

In some embodiments, a method of producing isopropanol is provided which comprises culturing any of the recombinant microorganisms of the present disclosure for a time under aerobic conditions or micro-aerobic conditions, to produce a cell mass, in particular in the range of from about 1 to about 100 g dry cells liter, or preferably in the range of from about 1 to about 10 g dry cells liter⁻¹, then altering the culture conditions for a time and under conditions to produce isopropanol, in particular for a time and under conditions wherein isopropanol is detectable in the culture, and recovering isopropanol. In certain embodiments, the culture conditions are altered from aerobic or micro-aerobic conditions to anaerobic conditions. In certain embodiments, the culture conditions are altered from aerobic conditions to micro-aerobic conditions.

Methods for recovering the isopropanol produced are well known to those skilled in the art. For example, isopropanol may be isolated from the culture medium by methods, such as pervaporation, liquid-liquid extraction, or gas stripping.

In certain embodiments, the engineered microorganism produces isopropanol at a yield of greater than 40% of theoretical, a volumetric productivity of greater than 0.2 g/l/h and a final titer of greater than 5 g/l isopropanol.

In certain embodiments, the engineered microorganism produces isopropanol at a yield of greater than 50% of theoretical, a volumetric productivity of greater than 0.4 g/l/h and a final titer of greater than 14 g/l isopropanol.

In certain embodiments, a recombinant microorganism herein described that expresses a pathway for the production of isopropanol, is further engineered to inactivate any competing pathways that consume metabolic intermediates of the isopropanol producing pathway. In other words the recombinant microorganism is further engineered to direct the carbon flux from the carbon source to isopropanol. In particular, direction of carbon-flux to isopropanol can be performed by inactivating metabolic pathways that compete with the isopropanol production pathway.

In certain embodiments, inactivation of a competing pathway is performed by inactivating an enzyme involved in the conversion of a substrate to a product within the competing pathway. The enzyme that is inactivated may preferably catalyze the conversion of a metabolic intermediate for the production of isopropanol or may catalyze the conversion of a metabolic intermediate of the competing pathway.

Accordingly, in certain embodiments the inactivation is performed by deleting from the microorganism's genome a gene coding for an enzyme involved in pathway that competes with the isopropanol production to make available the carbon to the one or more enzymes of the isopropanol producing pathway.

In certain embodiments, deletion of the genes encoding for these enzymes improves the isopropanol yield because more carbon is made available to one or more enzymes of the isopropanol producing pathway.

It will be appreciated by those skilled in the art that various omissions, additions and modifications may be made to the invention described above without departing from the scope of the invention, and all such modifications and changes are intended to fall within the scope of the invention, as defined by the appended claims.

### EXAMPLE 1

### Materials and Methods

Constructs

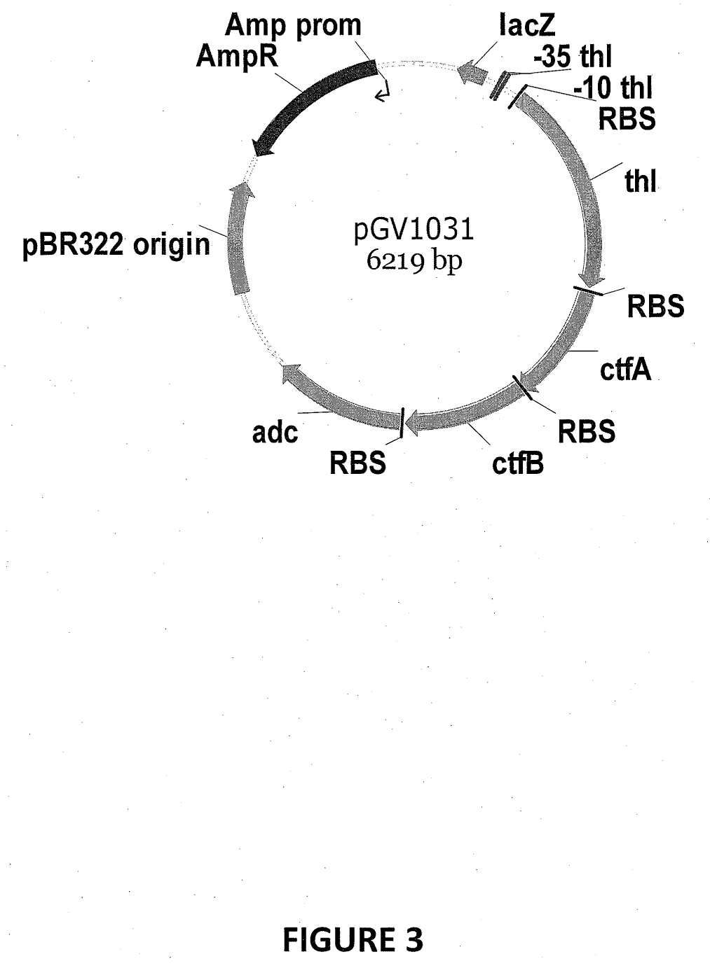

pGV1031: E. coli cells transformed with plasmid pACT, also referred to herein as plasmid pGV1031 were used to convert glucose to acetone. The plasmid contains the thl, ctfA, ctfB, and adc genes under the control of the native thiolase promoter. Plasmid pACT has been described previously (Bermejo L L, Welker N E, Papoutsakis E T, Expression of Clostridium acetobutylicum ATCC 824 genes in Escherichia coli for acetone production and acetate detoxification, Appl Environ Microbiol, 64(3):1079-85 (1998 March). thl encodes the thiolase enzyme that catalyzes the condensation reaction of two acetyl CoA molecules to generate acetoacetyl-CoA. ctfA and ctfB encode subunits of acetoacetyl-CoA:acetate/butyrate CoA tranferase (CoAT) that converts the acetoacetyl-CoA and acetic/butyric acid into acetoacetate and the corresponding acyl-CoA. adc encodes the acetoacetate decarboxylase that catalyzes the conversion of acetoacetate to acetone and carbon dioxide. Plasmid pGV1031 is shown in FIG. 3 and its sequence is given in SEQ ID NO:1.

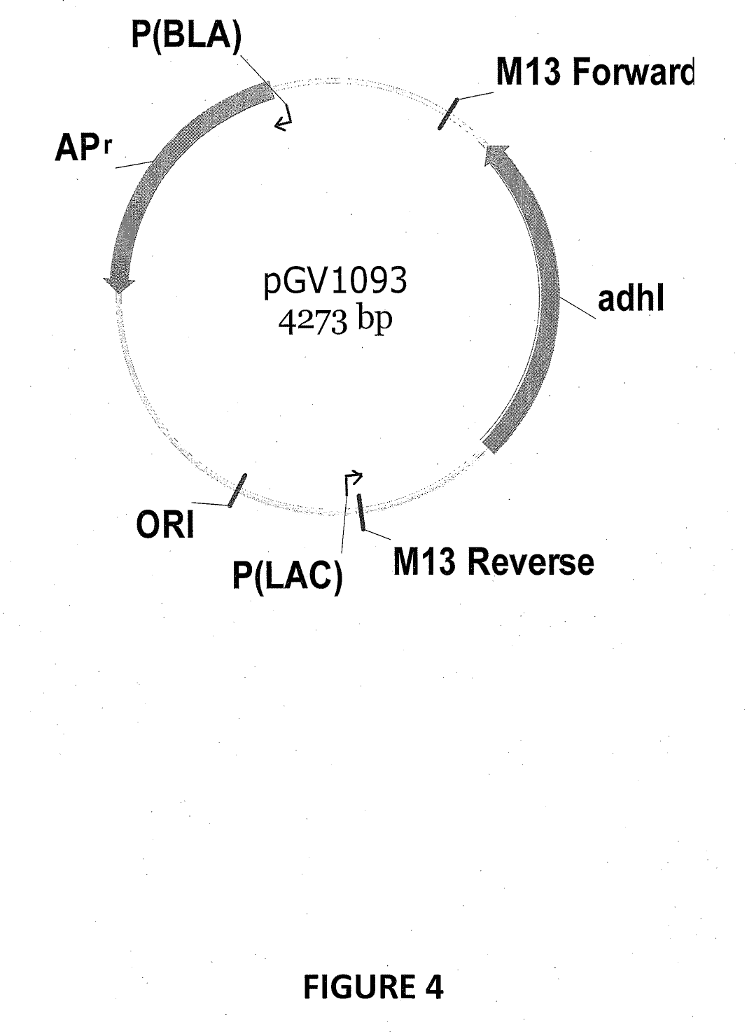

pGV1093: E. coli cells transformed with plasmid pGV1093 were used to convert acetone to isopropanol. This plasmid contains the gene for the primary/secondary alcohol dehydrogenase (adhI) from the Clostridium beijerinckii strain NRRL B593. Plasmid pGV1093 was derived from the previously described pGL89 plasmid (Peretz M, Bogin O, Tel-Or S, Cohen A, Li G, Chen J S, Burstein Y. Molecular Cloning, Nucleotide Sequencing, and Expression of Genes Encoding Alcohol Dehydrogenases From the Thermophile Thermoanaerobacter brockii and the Mesophile Clostridium beijerinckii, Anaerobe, 3(4):259-70) (August 1997). pGV1093 was constructed by subcloning an approximately 1.6 kb EcoRI/BamHI fragment containing adhI from pGL89 into pUC19 digested with EcoRI/BamHI. pGV1093 is shown in FIG. 4 and its sequence is given in SEQ ID NO:2.

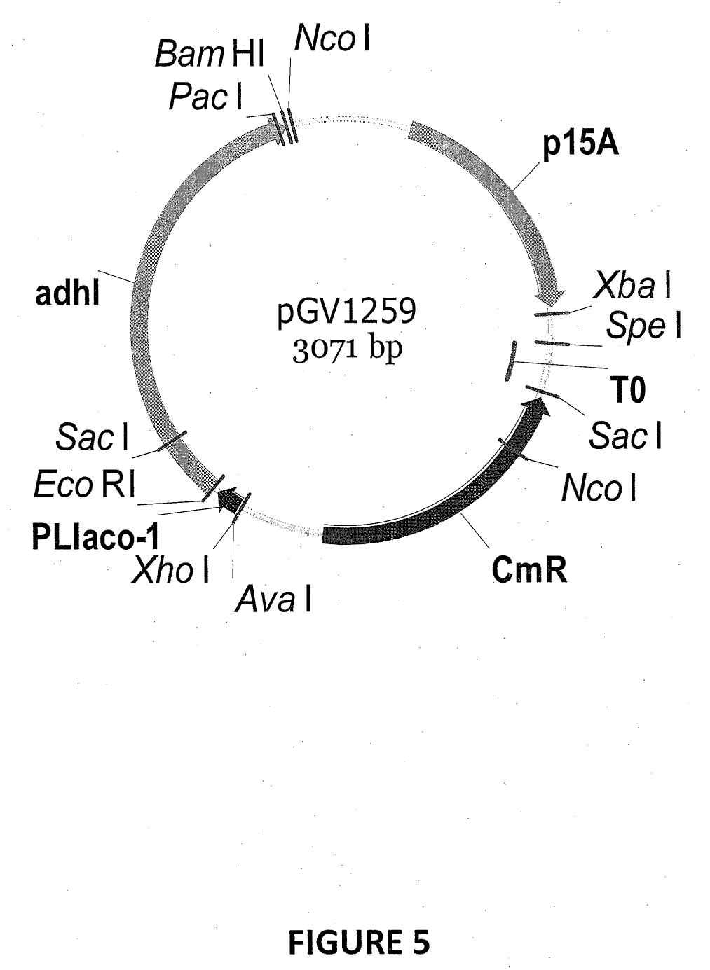

pGV1259: To convert glucose to isopropanol directly, five genes are co-expressed from two separate plasmids. These are: a primary/secondary alcohol dehydrogenase from Clostridium beijerinckii, herein referred to as adhI; thl, a gene encoding thiolase from Clostridium acetobutylicum; ctfA and ctfB, the genes encoding acetoacetyl-CoA:acetate/butyrate coenzyme-A transferase subunits from C. acetobutylicum; and adc, the gene encoding acetoacetate decarboxylase from C. acetobutylicum. pGV1093, the plasmid expressing adhI is not preferred for co-transformation into E. coli with pACT for two reasons: 1) both plasmids have a ColE1 origin of replication, and 2) both plasmids contain an ampicillin resistance marker for plasmid maintenance. To solve these problems and in order to co-express adhI and the genes on pGV1031, adhI is subcloned from pGV1093 into a more suitable expression vector, pZA32 (Lutz and Bujard, Nucleic Acids Res., 25(6): 1203-1210, 1997). pZA32 has a p15A origin of replication, a chloramphenicol resistance marker for plasmid maintenance, and P_{LlacO-1} promoter for adhI expression. The adhI gene is PCR amplified from pGV1093 using primers 487 (5′-AATTGGCGCCGAATTCATGAAAGGTTTTGC-3′) and 488 (5′-AATTCCCGGGGGATCCTAATATAACTACTG-3′) containing EcoRI and BamHI restriction sites in the forward and reverse primers, respectively. The amplified PCR product and pZA32 are digested with the restriction enzymes EcoRI and BamHI, gel purified, and then ligated together. The resulting plasmid, pGV1259, expresses adhI from the P_{LlacO-1} promoter. The plasmid map of pGV1259 is depicted in FIG. 5, the sequence is given in SEQ ID NO:3.

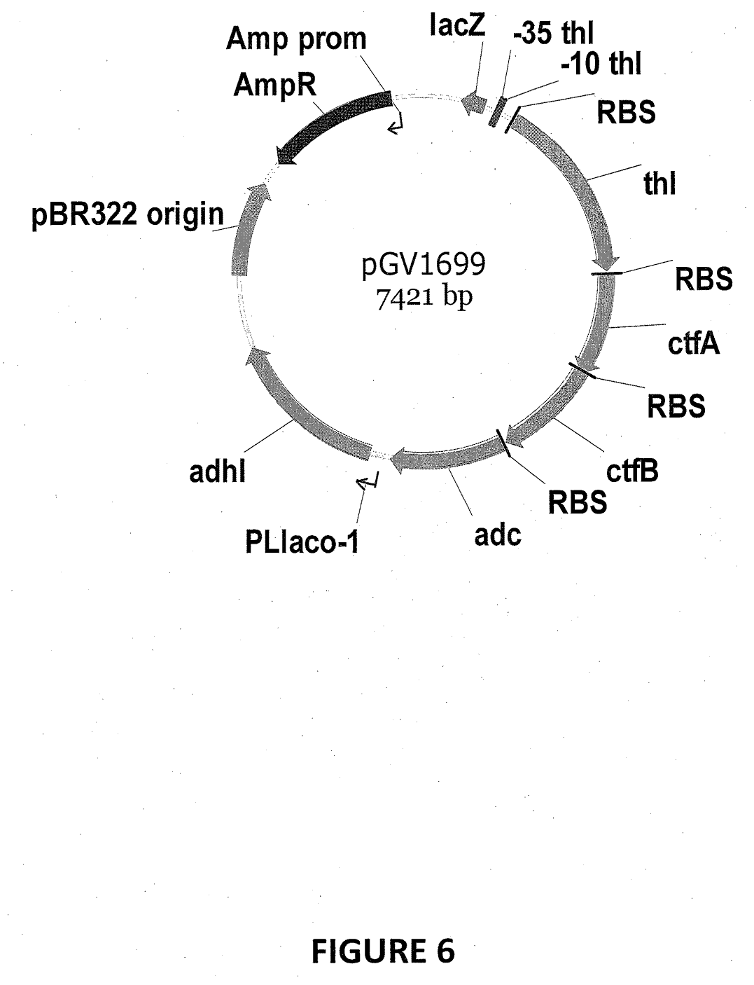

pGV1699: As an alternative to pGV1259 plasmid pGV1699 is designed which expresses all five genes of pathway 1 on a single plasmid. The nucleotide sequence encoding for P_{LlacO-1} and adhI is PCR amplified from pGV1259 using primers 1246 (5′-AATTGTCGACCGAGAAATGTGAGCGGATAAC-3′) and 1247 (5′-AATTGCATGCGTCTTTCGACTGAGCCTTTCG-3′) containing SalI and SphI, respectively. The amplified PCR product and pGV1031 are restriction digested using enzymes SalI and SphI, gel purified, and then ligated together using the Rapid Ligation Kit (Roche, Indianapolis, Ind.). The resulting plasmid expresses the C. acetobutylicum thl, ctfA/B, adc genes from the native thl promoter and the C. beijerinckii adhI from the P_{LlacO-1} promoter. The plasmid map of pGV1699 is depicted in FIG. 6 and its sequence is given in SEQ ID NO:9.

### EXAMPLE 2

### In Vivo Acetone Production in E. coli Using C. acetobutylicum Genes

Transformation and Cell Growth. Electrocompetent E. coli W3110 (GenBank: AP009048), E. coli B (GenBank: AAWW00000000) and E. coli ER2275 (Bermejo et al., Appl. Environ. Microbiol., 64(3): 1079-1085, 1998) cells were freshly transformed with pGV1031 and plated onto LB-ampicillin 100 μg/mL plates for 12 hrs at 37° C. Single colonies from the LB-ampicillin plates were used to inoculate 5 mL cultures of SD-7 medium (Luli and Strohl, Appl. Environ. Microbiol., 56(4), 1004-1011, 1990) containing 100 μg/mL ampicillin and allowed to grow for 12 hrs at 37° C. at 250 rpm. The above precultures were used to inoculate 125 mL of SD-8 medium (Luli and Strohl, Appl. Environ. Microbiol., 64(3), 1004-1011, 1990) containing 100 μg/mL ampicillin in 2 L Erlenmeyer flasks at 1% (vol/vol) of inoculum. Cultures were grown at 37° C. and 250 rpm. 3 mL samples were taken from the cultures every 3 hrs for 30 hrs with the first sample taken at the time of inoculation. Samples were used to monitor acetone and acetate production by gas chromatography (GC) and liquid chromatography (LC).

Product analysis. Samples were prepared for GC analysis by centrifuging the 3 mL aliquots at 5000×g for 10 min, followed by filtration through a 0.2 μm filter. A volume of 900 μL of the sample was transferred to a 1.5 mL gas chromatography vial and 90 μL of 10 mM 1-butanol was added as an internal standard. Samples were run on a Series II Plus gas chromatograph with a flame ionization detector (FID), fitted with a HP-7673 autosampler system using purchased standards and 5-point calibration curves with internal standards. All samples were injected at a volume of 1.0 μL. Direct analysis of the acetone product was performed on a Supelco SPB-1 capillary column (60 m length, 0.53 mm ID, 5 μm film thickness) connected to the FID detector. The temperature program for separating the products was 225° C. injector, 225° C. detector, 50° C. oven for 3 minutes, then 8° C./minute gradient to 80° C., 13° C./minute gradient to 170° C., 50° C./minute gradient to 220° C. then 220° C. for 3 minutes.

The samples were also analyzed by LC to monitor acetate production. A 900 μL volume of the filtered samples was transferred to 1.5 ml vial. The samples were run on an Aminex HPX-87H column using a 0.008N sulfuric acid mobile phase at a flow rate of 0.05 mL/min. The total run time was 30 min.

Results. The results shown in Table 1 demonstrate that acetone was produced in all three strains of E. coli tested carrying plasmid pGV1031. These experiments also demonstrate that acetone starts to accumulate 8-10 hrs after inoculation.

TABLE 1
Acetone production in E. coli strains transformed with pGV1031

Yield

| | | Acetone | Acetate |
|---|---|---|---|
| Strain | Time [hrs] | [mM] | [mM] |
| E. coli W3110 | 30 | 131 | 3.6 |
| (pGV1031) | | | |
| E. coli B | 30 | 112 | 9.7 |
| (pGV1031) | | | |
| E. Coli ER2275 | 30 | 151 | 91 |
| (pGV1031) | | | |

### EXAMPLE 3

### Heterologous Expression of the C. beijerinckii adhI Gene in E. coli to Convert Acetone to Isopropanol

Transformation and Cell Growth. E. coli DH5α Z1 electrocompetent cells were freshly transformed with pGV1093. As a control, E. coli DH5α Z1 electrocompetent cells were freshly transformed with pUC19, which does not contain an alcohol dehydrogenase. The transformed cells were plated onto LB-Ampicillin 100 μg/mL plates and incubated for 12 hrs at 37° C. To grow the strains, 4 mL precultures of both E. coli DH5α Z1 pGV1093 and E. coli DH5α Z1 pUC19 in LB-Ampicillin 100 μg/ml were inoculated with single colonies of freshly transformed cells from the LB-Ampicillin plates. These cultures were grown overnight (approximately 18 h) at 37° C. with shaking at 250 rpm. Precultures were used to inoculate 15 mL LB-Ampicillin 100 μg/mL cultures in 250 mL Erlenmeyer flasks at a rate of 1% (vol/vol) of the preculture used as inoculum. Cultures were grown at 37° C. with shaking at 250 rpm for 5 hrs, then induced with 1 mM IPTG (isopropyl-β-D-thiogalactopyranoside). To initiate the biotransformation, 96 μL of acetone was added to each of the cultures. The cultures were then further incubated at 30° C. at 250 rpm. To determine protein expression levels and to measure isopropanol and acetone concentrations, 1 mL samples were removed from each of the cultures prior to acetone addition, directly after the acetone addition, and 3 hrs after acetone addition.

Product analysis. Samples were prepared for GC analysis of isopropanol and acetone content by centrifuging the 1 mL aliquots at 5000×g for 10 min, followed by filtration through a 0.2 μm filter. A 900 μL volume of the sample was transferred to a 1.5 mL gas chromatography vial and 90 μL of 10 mM 1-butanol was added as an internal standard. Samples were run on a Series II Plus gas chromatograph with a flame ionization detector (FID), fitted with a HP-7673 autosampler system using purchased standards and 5-point calibration curves with internal standards. All samples were injected at a volume of 1.0 μL. Direct analysis of the acetone substrate and the isopropanol product was performed on a Supelco SPB-1 capillary column (60 m length, 0.53 mm ID, 5 μm film thickness) connected to the FID detector. The temperature program for separating the alcohol products was 225° C. injector, 225° C. detector, 50° C. oven for 3 minutes, then 15° C./minute gradient to 115° C., 25° C./minute gradient to 225° C., then 250° C. for 3 minutes.

Results. The results shown in Table 2 demonstrate that isopropanol was produced in E. coli containing pGV1093 but not pUC19.

TABLE 2
Isopropanol production in E. coli
strains transformed with pGV1093

| | | | approx. concentration | |
|---|---|---|---|---|
| | | | acetone | isopropanol |
| Sample | Strain | time | [mM] | [μM] |
| Control | E. coli DH5α Z1 | pre acetone | 0 | 0 |
| | (pUC19) | addition | | |
| | E. coli DH5α Z1 | directly post | 124 | 0 |
| | (pUC19) | acetone addition | | |
| | E. coli DH5α Z1 | 3 hrs after | 123 | 0 |
| | (pUC19) | acetone addition | | |
| Reaction | E. coli DH5α Z1 | pre acetone | 0 | 0 |
| | (pGV1093) | addition | | |
| | E. coli DH5α Z1 | directly post | 112 | 18 |
| | (pGV1093) | acetone addition | | |
| | E. coli DH5α Z1 | 3 hrs after | 117 | 165 |
| | (pGV1093) | acetone addition | | |

### EXAMPLE 4

### In Vivo Isopropanol Production in E. coli Using C. acetobutylicum Genes and the C. beijerinckii adhI Gene Expressed from Two Plasmids

E. coli W3110Z1 (Lutz and Bujard, Nucleic Acids Res., 25(6): 1203-1210, 1997) electrocompetent cells are freshly co-transformed with pGV1259 and pGV1031. The transformed cells are plated onto LB-ampicillin 100 μg/mL, -chloramphenicol 25 μg/mL plates and incubated for 12 hrs at 37° C.

Single colonies from the transformed plates are used to inoculate 5 mL of SD-7 medium (Luli and Strohl, Appl. Environ. Microbiol., 56(4), 1004-1011, 1990) containing ampicillin 100 μg/mL and chloramphenicol 25 μg/mL. These cultures are incubated for 12 hrs at 37° C. at 250 rpm. The above precultures are used to inoculate 125 mL of SD-8 medium (Luli and Strohl, Appl. Environ. Microbiol., 56(4), 1004-1011, 1990) containing ampicillin 100 μg/mL and chloramphenicol 25 μg/mL in 2 L Erlenmeyer flasks at 1% (vol/vol) of inoculum.

Cultures are grown at 37° C. and growth is monitored by OD 600 nm every hour. The culture is induced with 1 mM isopropyl β-D-thiogalactoside (IPTG) during the late-exponential phase. To monitor isopropanol production, culture samples (3 mL) are taken from the cultures every 3 hrs for 30 hrs with the first sample taken at the time of inoculation. Samples are processed and analyzed by GC and LC for acetone and isopropanol production as described in Example 2 and Example 3.

The engineered microorganism is expected to produce isopropanol at a yield of greater than 40% of theoretical, a volumetric productivity of greater than 0.2 g/l/h and a final titer of greater than 5 g/l isopropanol.

Using this system, the thl, ctfA/B and adc genes are expressed constitutively from the native thiolase promoter whereas the adhI gene is expressed from the inducible P_{LlacO-1} promoter, to allow for initial acetone accumulation followed by production of isopropanol. This system allows the time of induction of the adhI gene to vary and then the corresponding isopropanol production to be monitored.

### EXAMPLE 5

### In Vivo Isopropanol Production in E. coli Using C. acetobutylicum Genes and the C. beijerinckii adhI Gene Expressed from a Single Plasmid

E. coli W3110 Z1 (Lutz and Bujard, Nucleic Acids Res., 25(6): 1203-1210, 1997) electrocompetent cells are freshly co-transformed with pGV1699, carrying genes thl, ctfA/B, adc expressed from the native C. acetobutylicum thl promoter and C. beijerinckii adhI, from a P_{LlacO-1} promoter. The transformed cells are plated onto LB-ampicillin 100 μg/mL plates and incubated for 12 hrs at 37° C.

Single colonies from the transformed plates are used to inoculate 5 mL of SD-7 medium (Luli and Strohl, Appl. Environ. Microbiol., 56(4), 1004-1011, 1990) containing ampicillin 100 μg/mL. These cultures are incubated for 12 hrs at 37° C. at 250 rpm. The above precultures are used to inoculate 125 mL of SD-8 medium (Luli and Strohl, Appl. Environ. Microbiol., 56(4), 1004-1011, 1990) containing ampicillin 100 μg/mL and chloramphenicol 25 μg/mL in 2 L Erlenmeyer flasks at 1% (vol/vol) of inoculum.

Cultures are grown at 37° C. and growth is monitored by OD 600 nm every hour. The culture is induced with 1 mM isopropyl β-D-thiogalactoside (IPTG) during the late-exponential phase. To monitor isopropanol production, culture samples (3 mL) are taken from the cultures every 3 hrs for 30 hrs with the first sample taken at the time of inoculation. Samples are processed and analyzed by GC and LC for acetone and isopropanol production as described in Example 2 and Example 3.

The engineered microorganism is expected to produce isopropanol at a yield of greater than 50% of theoretical, a volumetric productivity of greater than 0.4 g/l/h and a final titer of greater than 14 g/l isopropanol.

1. A recombinant microbial host cell comprising each of the DNA molecules encoding a polypeptide or group of polypeptides that catalyze the conversion:
(i) Acetyl-CoA to Acetate and CoA (conversion 1)
(ii) Acetyl-CoA to Acetoacetyl-CoA and CoA (conversion 2)
(iii) Acetoacetyl-CoA and Acetate to Acetoacetate and Acetyl-CoA (conversion 3.1)
(iv) Acetoacetate to Acetone and CO2 (conversion 4)
(v) Acetone and NAD(P)H and H+ to Isopropanol and NAD(P)+ (conversion 5)
wherein the at least one DNA molecule is heterologous to said microbial host cell and wherein said microbial host cell produces isopropanol.

2. A host cell according to claim 1, wherein the host cell produces isopropanol at a yield of greater than 25% of theoretical.

3. A host cell according to claim 1, wherein the host cell produces isopropanol at a yield of greater than 40% of theoretical.

4. A host cell according to claim 1, wherein the host cell produces isopropanol at a yield of greater than 50% of theoretical.

5. A host cell according to claim 1, wherein the host cell produces isopropanol at a yield of greater than 75% of theoretical.

6. A host cell according to claim 1, wherein the group of polypeptides that catalyzes conversion 1 consists of phosphate acetyltransferase and acetate kinase.

7. A host cell according to claim 6, wherein the phosphate acetyltransferase is encoded by the E. coli gene pta and wherein the acetate kinase is encoded by the E. coli gene ackAB.

8. A host cell according to claim 1, wherein the polypeptide that catalyzes conversion 2 is acetyl-CoA-acetyltransferase.

9. A host cell according to claim 8, wherein the acetyl-CoA acetyltransferase has an amino acid sequence of SEQ ID NO:4.

10. A host cell according to claim 1, wherein the polypeptide that catalyzes conversion 3.1 is acetoacetyl-CoA:acetate/butyrate coenzyme-A transferase.

11. A host cell according to claim 10, wherein the acetoacetyl-CoA:acetate/butyrate coenzyme-A transferase is encoded by the C. acetobutyrlicum genes ctfA and ctfB which have corresponding amino acid sequence of SEQ ID NO:5 and 6.

12. A host cell according to claim 1, wherein the polypeptide that catalyzes conversion 4 is acetoacetate decarboxylase.

13. A host cell according to claim 12, wherein the acetoacetate decarboxylase has an amino acid sequence of SEQ ID NO:7.

14. A host cell according to claim 1, wherein the polypeptide that catalyzes conversion 5 is a secondary alcohol dehydrogenase.

15. A host cell according to claim 14, wherein said secondary alcohol dehydrogenase is heterologous to said microorganism.

16. A host cell according to claim 14, wherein said secondary alcohol dehydrogenase is not heterologous to said microorganism.

17. A host cell according to claim 14, wherein said secondary alcohol dehydrogenase is from Clostridium beijerinckii, from Burkholderia spp., or from Thermoanaerobacter brockii.

18. A host cell according to claim 17, wherein said Clostridium beijerinckii is strain NRRL B593 or strain NESTE 225.

19. A host cell according to claim 14, wherein said alcohol dehydrogenase has an amino acid sequence of SEQ ID NO:8.

20. A host cell according to claim 1, wherein said microorganism comprises deletion or inactivation of competing acetyl-CoA consuming genes.

21. A host cell according to claim 1, wherein said microorganism is an E. coli strain which comprises deletion or inactivation of a gene or genes selected from the group consisting of poxB, adhE, ldhA, frdABCD, succinate dehydrogenase, malate dehydrogenase, alpha-ketoglutarate dehydrogenase and combinations thereof.

22. A host cell according to claim 1, wherein said microorganism is an E. coli strain which comprises deletion or inactivation of a gene or genes selected from the group consisting of poxB, ldhA, frdABCD and combinations thereof.

23. A host cell according to claim 1, wherein the cell is selected from the group consisting of: a bacterium, a cyanobacterium, a filamentous fungus and a yeast.

24. A host cell according to claim 1, wherein said microorganism is an E. coli.

25. A host cell according to claim 1, wherein said microorganism is a Saccharomyces cerevisiae.

26. A host cell according to claim 1, wherein said microorganism is a member of the genus Salmonella.

27. A host cell according to claim 1, wherein said microorganism is a member of the genus Bacillus.

28. A host cell according to claim 1, wherein said microorganism is a member of the genus Clostridium.

29. A host cell according to claim 1, wherein said microorganism is a member of a genus selected from the group consisting of Pichia, Hansenula, Yarrowia, Aspergillus, Kluyveromyces, Pachysolen, Rhodotorula, Zygosaccharomyces, Galactomyces, Schizosaccharomyces, Torulaspora, Debaryomyces, Williopsis, Dekkera, Kloeckera, Metschnikowia or Candida.

30. A host cell according to claim 1, wherein said microorganism is a member of a genus selected from the group consisting of Arthrobacter, Bacillus, Brevibacterium, Clostridium, Corynebacterium, Gluconobacter, Nocardia, Pseudomonas, Rhodococcus, Streptomyces, or Xanthomonas.

31. A host cell according to claim 1, wherein all of said polypeptides are heterologous to said microorganism.

32. A recombinant microbial host cell comprising each of the DNA molecules encoding a polypeptide that catalyzes the conversion:
(i) Acetyl-CoA to Acetoacetyl-CoA and CoA (conversion 2)
(ii) Acetoacetyl-CoA+H2O→Acetoacetate+CoA (conversion 3.2)
(iii) Acetoacetate to Acetone and CO2 (conversion 4)
(iv) Acetone and NAD(P)H and H+ to Isopropanol and NAD(P)+ (conversion 5)
wherein the at least one DNA molecule is heterologous to said microbial host cell and wherein said microbial host cell produces isopropanol.

33-59. (canceled)

60. A method for the production of isopropanol comprising:
(a) providing a recombinant microbial host cell comprising each of the DNA molecules encoding a polypeptide or group of polypeptides that catalyze the conversion:
(i) Acetyl-CoA to Acetate and CoA (conversion 1)
(ii) Acetyl-CoA to Acetoacetyl-CoA and CoA (conversion 2)
(iii) Acetoacetyl-CoA and Acetate to Acetoacetate and Acetyl-CoA (conversion 3.1)
(iv) Acetoacetate to Acetone and CO2 (conversion 4)
(v) Acetone and NAD(P)H and H+ to Isopropanol and NAD(P)+ (conversion 5)
wherein the at least one DNA molecule is heterologous to said microbial host cell;
(b) contacting the host cell of (i) with a fermentable carbon substrate in a fermentation medium under conditions whereby isopropanol is produced; and
(c) recovering said isopropanol.

61-84. (canceled)

85. An isopropanol containing fermentation medium produced by a method comprising:
(a) providing recombinant microbial host cell comprising each of the DNA molecules encoding a polypeptide or group of polypeptides that catalyze the conversion:
(i) Acetyl-CoA to Acetate and CoA (conversion 1)
(ii) Acetyl-CoA to Acetoacetyl-CoA and CoA (conversion 2)
(iii) Acetoacetyl-CoA and Acetate to Acetoacetate and Acetyl-CoA (conversion 3.1)
(iv) Acetoacetate to Acetone and CO2 (conversion 4)
(v) Acetone and NAD(P)H and H+ to Isopropanol and NAD(P)+ (conversion 5)
wherein the at least one DNA molecule is heterologous to said microbial host cell;
(b) contacting the host cell of (i) with a fermentable carbon substrate in a fermentation medium under conditions whereby isopropanol is produced; and
(c) recovering said isopropanol.

86. Isopropanol produced by a method comprising:
(a) providing recombinant microbial host cell comprising each of the DNA molecules encoding a polypeptide or group of polypeptides that catalyze the conversion:
(i) Acetyl-CoA to Acetate and CoA (conversion 1)
(ii) Acetyl-CoA to Acetoacetyl-CoA and CoA (conversion 2)
(iii) Acetoacetyl-CoA and Acetate to Acetoacetate and Acetyl-CoA (conversion 3.1)
(iv) Acetoacetate to Acetone and CO2 (conversion 4)
(v) Acetone and NAD(P)H and H+ to Isopropanol and NAD(P)+ (conversion 5)
wherein the at least one DNA molecule is heterologous to said microbial host cell;
(b) contacting the host cell of (i) with a fermentable carbon substrate in a fermentation medium under conditions whereby isopropanol is produced; and
(c) recovering said isopropanol.

87. A method for the production of isopropanol comprising:
(a) providing a recombinant microbial host cell comprising each of the DNA molecules encoding a polypeptide that catalyzes the conversion:
(i) Acetyl-CoA to Acetoacetyl-CoA and CoA (conversion 2)
(ii) Acetoacetyl-CoA+H2O→Acetoacetate+CoA (conversion 3.2)
(iii) Acetoacetate to Acetone and CO2 (conversion 4)
(iv) Acetone and NAD(P)H and H+ to Isopropanol and NAD(P)+ (conversion 5)
wherein the at least one DNA molecule is heterologous to said microbial host cell;
(b) contacting the host cell of (i) with a fermentable carbon substrate in a fermentation medium under conditions whereby isopropanol is produced; and
(c) recovering said isopropanol.

88-108. (canceled)

109. An isopropanol containing fermentation medium produced by a method comprising:
(a) providing a recombinant microbial host cell comprising each of the DNA molecules encoding a polypeptide that catalyzes the conversion:
(i) Acetyl-CoA to Acetoacetyl-CoA and CoA (conversion 2)
(ii) Acetoacetyl-CoA+H2O→Acetoacetate+CoA (conversion 3.2)
(iii) Acetoacetate to Acetone and CO2 (conversion 4)
(iv) Acetone and NAD(P)H and H+ to Isopropanol and NAD(P)+ (conversion 5)
wherein the at least one DNA molecule is heterologous to said microbial host cell;
(b) contacting the host cell of (i) with a fermentable carbon substrate in a fermentation medium under conditions whereby isopropanol is produced; and
(c) recovering said isopropanol.

110. Isopropanol produced by a method comprising:
(a) providing a recombinant microbial host cell comprising each of the DNA molecules encoding a polypeptide that catalyzes the conversion:
(i) Acetyl-CoA to Acetoacetyl-CoA and CoA (conversion 2)
(ii) Acetoacetyl-CoA+H2O→Acetoacetate+CoA (conversion 3.2)
(iii) Acetoacetate to Acetone and CO2 (conversion 4)
(iv) Acetone and NAD(P)H and H+ to Isopropanol and NAD(P)+ (conversion 5)
wherein the at least one DNA molecule is heterologous to said microbial host cell;
(b) contacting the host cell of (i) with a fermentable carbon substrate in a fermentation medium under conditions whereby isopropanol is produced; and
(c) recovering said isopropanol.


SEQUENCE LISTING (9 sequences)

SEQ ID NO: 1 - DNA, 6219 bp, Artificial
  Plasmid pGV1031
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtaccatatgcataagtttaatttttttgttaaaaaatattaaactttgtgttttttttaacaaaatatattgataaaaataataatagtgggtataattaagttgttagagaaaacgtataaattagggataaactatggaacttatgaaatagattgaaatggtttatctgttaccccgtatcaaaatttaggaggttagttagaatgaaagaagttgtaatagctagtgcagtaagaacagcgattggatcttatggaaagtctcttaaggatgtaccagcagtagatttaggagctacagctataaaggaagcagttaaaaaagcaggaataaaaccagaggatgttaatgaagtcattttaggaaatgttcttcaagcaggtttaggacagaatccagcaagacaggcatcttttaaagcaggattaccagttgaaattccagctatgactattaataaggtttgtggttcaggacttagaacagttagcttagcagcacaaattataaaagcaggagatgctgacgtaataatagcaggtggtatggaaaatatgtctagagctccttacttagcgaataacgctagatggggatatagaatgggaaacgctaaatttgttgatgaaatgatcactgacggattgtgggatgcatttaatgattaccacatgggaataacagcagaaaacatagctgagagatggaacatttcaagagaagaacaagatgagtttgctcttgcatcacaaaaaaaagctgaagaagctataaaatcaggtcaatttaaagatgaaatagttcctgtagtaattaaaggcagaaagggagaaactgtagttgatacagatgagcaccctagatttggatcaactatagaaggacttgcaaaattaaaacctgccttcaaaaaagatggaacagttacagctggtaatgcatcaggattaaatgactgtgcagcagtacttgtaatcatgagtgcagaaaaagctaaagagcttggagtaaaaccacttgctaagatagtttcttatggttcagcaggagttgacccagcaataatgggatatggacctttctatgcaacaaaagcagctattgaaaaagcaggttggacagttgatgaattagatttaatagaatcaaatgaagcttttgcagctcaaagtttagcagtagcaaaagatttaaaatttgatatgaataaagtaaatgtaaatggaggagctattgcccttggtcatccaattggagcatcaggtgcaagaatactcgttactcttgtacacgcaatgcaaaaaagagatgcaaaaaaaggcttagcaactttatgtataggtggcggacaaggaacagcaatattgctagaaaagtgctagaaaggatccagaatttaaaaggagggattaaaatgaactctaaaataattagatttgaaaatttaaggtcattctttaaagatgggatgacaattatgattggaggttttttaaactgtggcactccaaccaaattaattgattttttagttaatttaaatataaagaatttaacgattataagtaatgatacatgttatcctaatacaggtattggtaagttaatatcaaataatcaagtaaaaaagcttattgcttcatatataggcagcaacccagatactggcaaaaaactttttaataatgaacttgaagtagagctctctccccaaggaactctagtggaaagaatacgtgcaggcggatctggcttaggtggtgtactaactaaaacaggtttaggaactttgattgaaaaaggaaagaaaaaaatatctataaatggaacggaatatttgttagagctacctcttacagccgatgtagcattaattaaaggtagtattgtagatgaggccggaaacaccttctataaaggtactactaaaaactttaatccctatatggcaatggcagctaaaaccgtaatagttgaagctgaaaatttagttagctgtgaaaaactagaaaaggaaaaagcaatgacccccggagttcttataaattatatagtaaaggagcctgcataaaatgattaatgataaaaacctagcgaaagaaataatagccaaaagagttgcaagagaattaaaaaatggtcaacttgtaaacttaggtgtaggtcttcctaccatggttgcagattatataccaaaaaatttcaaaattactttccaatcagaaaacggaatagttggaatgggcgctagtcctaaaataaatgaggcagataaagatgtagtaaatgcaggaggagactatacaacagtacttcctgacggcacatttttcgatagctcagtttcgttttcactaatccgtggtggtcacgtagatgttactgttttaggggctctccaggtagatgaaaagggtaatatagccaattggattgttcctggaaaaatgctctctggtatgggtggagctatggatttagtaaatggagctaagaaagtaataattgcaatgagacatacaaataaaggtcaacctaaaattttaaaaaaatgtacacttcccctcacggcaaagtctcaagcaaatctaattgtaacagaacttggagtaattgaggttattaatgatggtttacttctcactgaaattaataaaaacacaaccattgatgaaataaggtctttaactgctgcagatttactcatatccaatgaacttagacccatggctgtttagaaagaattcttgatatcaggaaggtgacttttatgttaaaggatgaagtaattaaacaaattagcacgccattaacttcgcctgcatttcctagaggaccctataaatttcataatcgtgagtattttaacattgtatatcgtacagatatggatgctcttcgtaaagttgtgccagagcctttagaaattgatgagcccttagtcaggtttgaaattatggcaatgcatgatacgagtggacttggttgttatacagaaagcggacaggctattcccgtaagctgtaatggagttaagggagattatcttcatatgatgtatttagataatgagcctgcaattgcagtaggaagggaattaagtgcatatcctaaaaagctcgggtatccaaagctttttgtggattcagatactttagtaggaactttagactatggaaaacttagagttgcgacagctacaatggggtacaaacataaagccttagatgctaatgaagcaaaggatcaaatttgtcgccctaattatatgttgaaaataatacccaattatgatggaagccctaggatatgtgagcttataaatgcgaaaatcacagatgttaccgtacatgaagcttggacaggaccaactcgactgcagttatttgatcacgctatggcgccacttaatgatttgccagtaaaagagattgtttctagctctcacattcttgcagatataatattgcctagagctgaagttatatatgattatcttaagtaataaaaataagagttaccttaaatggtaactcttatttttttaatgtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 2 - DNA, 4273 bp, Artificial
  Plasmid pGV1093
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattctatgataataaactgtccaggctttgcagattttgctactcttggagcttctatatccattgagaatatattgtttgttagctcctttttactaactatcttgtacatgtataatcctccatgatctattatgttataatataactactgctttaattaagtcttttggcttatctttcattaataacagtgcttcttctatgtgatcaaatccatgatatacatgtgtaactaatttacttagatcaacacgattatatactaccatatctcttaacatttctgctctcaaacgtcccccaggacaaagacctccttttatagtcttgtgagccattccacatccccattctacacgtggtattagtaaagcatctccacttccatgataatttatattagaaattattcctcctggtttaaccatagatactgcttgggataatgtttcagaaccaccgcctgccataattacgcggtcaacgccttttccattcgttaatttcataacttgatcaactatatgaccatttttataatttagaatatctgttgctccataaaattttgcagcctcaacacaaatcggcctgctccccactccaattattctacctgctccacgtaatttagcacctgctattcccattaagccaacagctccaatgccaattaccacaacacttgaacccatttgaatatctgcaagttctgctccatgaaatccagtagtcatcatatctgttatcataacagcattttctaatggcatgtctttaggtagaatcgcaagattcatatccgcatcatttacatgaaaatattcaccaaaaactccatccttgaaatttgaaaatttccatcctgcgagcataccgtttgagtgctgttgaaaaccagcttgaacttccaaagatctccaatctggagttgtacaaggaactataactctgtcaccaggtttaaaatccttcacttcacttcctacttcaacaacttcacctacagcttcatgccctaaaatcatattcttcctatctccaagagctccctcaaaaacagtatgtatatctgatgtacacggagatactgctaatgggcgtacaatagcatcatatgaacccgcaactggcctttctttttcgatccatcctaacttattaatacctagcattgcaaaacctttcataaaatatgttcctccttaaaaatattcctttaatagtctaaaaacatcgttaaaaaattatttttaaaatttgttttagtcttatatgatatatttaagcaaatactatgccaaaatataatattttaaacatattatgaagttatattataactatctgcctgttcatcgctatttcgcttgagatataatataagcttttatgaataataatattattattcatattgaaccagaaatgctgttgaaaaaaacaacatttacatttccaataactgtcgtattttccgccagtgttgtattttcatacatgttttaaattattgattgttaaaaaatatccataaaatcatctgacttttatattatatttttttatctttatatatagtgtacttctgtttattcctaatggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 3 - DNA, 3071 bp, Artificial
  Plasmid pGV1259
ctaggggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgttactagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagaaatgtgagcggataacaattgacattgtgagcggataacaagatactgagcacatcagcaggacgcactgaccgggaattcatgaaaggttttgcaatgctaggtattaataagttaggatggatcgaaaaagaaaggccagttgcgggttcatatgatgctattgtacgcccattagcagtatctccgtgtacatcagatatacatactgtttttgagggagctcttggagataggaagaatatgattttagggcatgaagctgtaggtgaagttgttgaagtaggaagtgaagtgaaggattttaaacctggtgacagagttatagttccttgtacaactccagattggagatctttggaagttcaagctggttttcaacagcactcaaacggtatgctcgcaggatggaaattttcaaatttcaaggatggagtttttggtgaatattttcatgtaaatgatgcggatatgaatcttgcgattctacctaaagacatgccattagaaaatgctgttatgataacagatatgatgactactggatttcatggagcagaacttgcagatattcaaatgggttcaagtgttgtggtaattggcattggagctgttggcttaatgggaatagcaggtgctaaattacgtggagcaggtagaataattggagtggggagcaggccgatttgtgttgaggctgcaaaattttatggagcaacagatattctaaattataaaaatggtcatatagttgatcaagttatgaaattaacgaatggaaaaggcgttgaccgcgtaattatggcaggcggtggttctgaaacattatcccaagcagtatctatggttaaaccaggaggaataatttctaatataaattatcatggaagtggagatgctttactaataccacgtgtagaatggggatgtggaatggctcacaagactataaaaggaggtctttgtcctgggggacgtttgagagcagaaatgttaagagatatggtagtatataatcgtgttgatctaagtaaattagttacacatgtatatcatggatttgatcacatagaagaagcactgttattaatgaaagataagccaaaagacttaattaaagcagtagttatattaggatccgatccgatcccatggtacgcgtgctagaggcatcaaataaaacgaaaggctcagtcgaaagactgggcctttcgttttatctgttgtttgtcggtgaacgctctcctgagtaggacaaatccgccgccctagac

SEQ ID NO: 4 - protein, 392 aa, Artificial
  thl
MKEVVIASAVRTAIGSYGKSLKDVPAVDLGATAIKEAVKKAGIKPEDVNEVILGNVLQAGLGQNPARQASFKAGLPVEIPAMTINKVCGSGLRTVSLAAQIIKAGDADVIIAGGMENMSRAPYLANNARWGYRMGNAKFVDEMITDGLWDAFNDYHMGITAENIAERWNISREEQDEFALASQKKAEEAIKSGQFKDEIVPVVIKGRKGETVVDTDEHPRFGSTIEGLAKLKPAFKKDGTVTAGNASGLNDCAAVLVIMSAEKAKELGVKPLAKIVSYGSAGVDPAIMGYGPFYATKAAIEKAGWTVDELDLIESNEAFAAQSLAVAKDLKFDMNKVNVNGGAIALGHPIGASGARILVTLVHAMQKRDAKKGLATLCIGGGQGTAILLEKC

SEQ ID NO: 5 - protein, 218 aa, Artificial
  ctfA
MNSKIIRFENLRSFFKDGMTIMIGGFLNCGTPTKLIDFLVNLNIKNLTIISNDTCYPNTGIGKLISNNQVKKLIASYIGSNPDTGKKLFNNELEVELSPQGTLVERIRAGGSGLGGVLTKTGLGTLIEKGKKKISINGTEYLLELPLTADVALIKGSIVDEAGNTFYKGTTKNFNPYMAMAAKTVIVEAENLVSCEKLEKEKAMTPGVLINYIVKEPA

SEQ ID NO: 6 - protein, 221 aa, Artificial
  ctfB
MINDKNLAKEIIAKRVARELKNGQLVNLGVGLPTMVADYIPKNFKITFQSENGIVGMGASPKINEADKDVVNAGGDYTTVLPDGTFFDSSVSFSLIRGGHVDVTVLGALQVDEKGNIANWIVPGKMLSGMGGAMDLVNGAKKVIIAMRHTNKGQPKILKKCTLPLTAKSQANLIVTELGVIEVINDGLLLTEINKNTTIDEIRSLTAADLLISNELRPMAV

SEQ ID NO: 7 - protein, 244 aa, Artificial
  adc
MLKDEVIKQISTPLTSPAFPRGPYKFHNREYFNIVYRTDMDALRKVVPEPLEIDEPLVRFEIMAMHDTSGLGCYTESGQAIPVSFNGVKGDYLHMMYLDNEPAIAVGRELSAYPKKLGYPKLFVDSDTLVGTLDYGKLRVATATMGYKHKALDANEAKDQICRPNYMLKIIPNYDGSPRICELINAKITDVTVHEAWTGPTRLQLFDHAMAPLNDLPVKEIVSSSHILADIILPRAEVIYDYLK

SEQ ID NO: 8 - protein, 351 aa, Artificial
  adhI
MKGFAMLGINKLGWIEKERPVAGSYDAIVRPLAVSPCTSDIHTVFEGALGDRKNMILGHEAVGEVVEVGSEVKDFKPGDRVIVPCTTPDWRSLEVQAGFQQHSNGMLAGWKFSNFKDGVFGEYFHVNDADMNLAILPKDMPLENAVMITDMMTTGFHGAELADIQMGSSVVVIGIGAVGLMGIAGAKLRGAGRIIGVGSRPICVEAAKFYGATDILNYKNGHIVDQVMKLTNGKGVDRVIMAGGGSETLSQAVSMVKPGGIISNINYHGSGDALLIPRVEWGCGMAHKTIKGGLCPGGRLRAEMLRDMVVYNRVDLSKLVTHVYHGFDHIEEALLLMKDKPKDLIKAVVIL

SEQ ID NO: 9 - DNA, 7421 bp, Artificial
  Plasmid pGV1699
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtaccatatgcataagtttaatttttttgttaaaaaatattaaactttgtgttttttttaacaaaatatattgataaaaataataatagtgggtataattaagttgttagagaaaacgtataaattagggataaactatggaacttatgaaatagattgaaatggtttatctgttaccccgtatcaaaatttaggaggttagttagaatgaaagaagttgtaatagctagtgcagtaagaacagcgattggatcttatggaaagtctcttaaggatgtaccagcagtagatttaggagctacagctataaaggaagcagttaaaaaagcaggaataaaaccagaggatgttaatgaagtcattttaggaaatgttcttcaagcaggtttaggacagaatccagcaagacaggcatcttttaaagcaggattaccagttgaaattccagctatgactattaataaggtttgtggttcaggacttagaacagttagcttagcagcacaaattataaaagcaggagatgctgacgtaataatagcaggtggtatggaaaatatgtctagagctccttacttagcgaataacgctagatggggatatagaatgggaaacgctaaatttgttgatgaaatgatcactgacggattgtgggatgcatttaatgattaccacatgggaataacagcagaaaacatagctgagagatggaacatttcaagagaagaacaagatgagtttgctcttgcatcacaaaaaaaagctgaagaagctataaaatcaggtcaatttaaagatgaaatagttcctgtagtaattaaaggcagaaagggagaaactgtagttgatacagatgagcaccctagatttggatcaactatagaaggacttgcaaaattaaaacctgccttcaaaaaagatggaacagttacagctggtaatgcatcaggattaaatgactgtgcagcagtacttgtaatcatgagtgcagaaaaagctaaagagcttggagtaaaaccacttgctaagatagtttcttatggttcagcaggagttgacccagcaataatgggatatggacctttctatgcaacaaaagcagctattgaaaaagcaggttggacagttgatgaattagatttaatagaatcaaatgaagcttttgcagctcaaagtttagcagtagcaaaagatttaaaatttgatatgaataaagtaaatgtaaatggaggagctattgcccttggtcatccaattggagcatcaggtgcaagaatactcgttactcttgtacacgcaatgcaaaaaagagatgcaaaaaaaggcttagcaactttatgtataggtggcggacaaggaacagcaatattgctagaaaagtgctagaaaggatccagaatttaaaaggagggattaaaatgaactctaaaataattagatttgaaaatttaaggtcattctttaaagatgggatgacaattatgattggaggttttttaaactgtggcactccaaccaaattaattgattttttagttaatttaaatataaagaatttaacgattataagtaatgatacatgttatcctaatacaggtattggtaagttaatatcaaataatcaagtaaaaaagcttattgcttcatatataggcagcaacccagatactggcaaaaaactttttaataatgaacttgaagtagagctctctccccaaggaactctagtggaaagaatacgtgcaggcggatctggcttaggtggtgtactaactaaaacaggtttaggaactttgattgaaaaaggaaagaaaaaaatatctataaatggaacggaatatttgttagagctacctcttacagccgatgtagcattaattaaaggtagtattgtagatgaggccggaaacaccttctataaaggtactactaaaaactttaatccctatatggcaatggcagctaaaaccgtaatagttgaagctgaaaatttagttagctgtgaaaaactagaaaaggaaaaagcaatgacccccggagttcttataaattatatagtaaaggagcctgcataaaatgattaatgataaaaacctagcgaaagaaataatagccaaaagagttgcaagagaattaaaaaatggtcaacttgtaaacttaggtgtaggtcttcctaccatggttgcagattatataccaaaaaatttcaaaattactttccaatcagaaaacggaatagttggaatgggcgctagtcctaaaataaatgaggcagataaagatgtagtaaatgcaggaggagactatacaacagtacttcctgacggcacatttttcgatagctcagtttcgttttcactaatccgtggtggtcacgtagatgttactgttttaggggctctccaggtagatgaaaagggtaatatagccaattggattgttcctggaaaaatgctctctggtatgggtggagctatggatttagtaaatggagctaagaaagtaataattgcaatgagacatacaaataaaggtcaacctaaaattttaaaaaaatgtacacttcccctcacggcaaagtctcaagcaaatctaattgtaacagaacttggagtaattgaggttattaatgatggtttacttctcactgaaattaataaaaacacaaccattgatgaaataaggtctttaactgctgcagatttactcatatccaatgaacttagacccatggctgtttagaaagaattcttgatatcaggaaggtgacttttatgttaaaggatgaagtaattaaacaaattagcacgccattaacttcgcctgcatttcctagaggaccctataaatttcataatcgtgagtattttaacattgtatatcgtacagatatggatgctcttcgtaaagttgtgccagagcctttagaaattgatgagcccttagtcaggtttgaaattatggcaatgcatgatacgagtggacttggttgttatacagaaagcggacaggctattcccgtaagctgtaatggagttaagggagattatcttcatatgatgtatttagataatgagcctgcaattgcagtaggaagggaattaagtgcatatcctaaaaagctcgggtatccaaagctttttgtggattcagatactttagtaggaactttagactatggaaaacttagagttgcgacagctacaatggggtacaaacataaagccttagatgctaatgaagcaaaggatcaaatttgtcgccctaattatatgttgaaaataatacccaattatgatggaagccctaggatatgtgagcttataaatgcgaaaatcacagatgttaccgtacatgaagcttggacaggaccaactcgactgcagttatttgatcacgctatggcgccacttaatgatttgccagtaaaagagattgtttctagctctcacattcttgcagatataatattgcctagagctgaagttatatatgattatcttaagtaataaaaataagagttaccttaaatggtaactcttatttttttaatgtcgaccgagaaatgtgagcggataacaattgacattgtgagcggataacaagatactgagcacatcagcaggacgcactgaccgggaattcatgaaaggttttgcaatgctaggtattaataagttaggatggatcgaaaaagaaaggccagttgcgggttcatatgatgctattgtacgcccattagcagtatctccgtgtacatcagatatacatactgtttttgagggagctcttggagataggaagaatatgattttagggcatgaagctgtaggtgaagttgttgaagtaggaagtgaagtgaaggattttaaacctggtgacagagttatagttccttgtacaactccagattggagatctttggaagttcaagctggttttcaacagcactcaaacggtatgctcgcaggatggaaattttcaaatttcaaggatggagtttttggtgaatattttcatgtaaatgatgcggatatgaatcttgcgattctacctaaagacatgccattagaaaatgctgttatgataacagatatgatgactactggatttcatggagcagaacttgcagatattcaaatgggttcaagtgttgtggtaattggcattggagctgttggcttaatgggaatagcaggtgctaaattacgtggagcaggtagaataattggagtggggagcaggccgatttgtgttgaggctgcaaaattttatggagcaacagatattctaaattataaaaatggtcatatagttgatcaagttatgaaattaacgaatggaaaaggcgttgaccgcgtaattatggcaggcggtggttctgaaacattatcccaagcagtatctatggttaaaccaggaggaataatttctaatataaattatcatggaagtggagatgctttactaataccacgtgtagaatggggatgtggaatggctcacaagactataaaaggaggtctttgtcctgggggacgtttgagagcagaaatgttaagagatatggtagtatataatcgtgttgatctaagtaaattagttacacatgtatatcatggatttgatcacatagaagaagcactgttattaatgaaagataagccaaaagacttaattaaagcagtagttatattaggatccgatccgatcccatggtacgcgtgctagaggcatcaaataaaacgaaaggctcagtcgaaagacgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc